(19) United States
(12) Patent Application Publication
(10) Pub. No.: US 2010/0316728 A1
(43) Pub. Date: Dec. 16, 2010

(54) Therapeutic mask and Method of Treatment Employing the Same

(76) Inventors: William H. Rreeves, Coral Springs, FL (US); Jonathan W. Reeves, Coral Springs, FL (US); Alejandro Rendon, Coconut Creek, FL (US); Marta J. Rendon, Boca Raton, FL (US)

Correspondence Address: A PATENT LAWYER CORP, PLC;R WILLIAM GRAHAM, 22 S ST CLAIR ST, DAYTON, OH, 45402, US

(21) Appl. No.: 12/484,306

(22) Filed: Jun. 15, 2009

(51) Int. Cl. A61K 35/24

(52) U.S. Cl. 424/537

(57) ABSTRACT

A therapeutic mask includes a therapeutic composition including honey and a penetrating substance, a dressing having said therapeutic composition combined therewith, wherein the dressing and the therapeutic composition are freeze dried to provide the therapeutic mask. There is also a method of making and using the composition.

### BACKGROUND OF THE INVENTION

1. Field of the Invention

The present invention relates to the field of dermal therapies. More particularly, the present invention is directed to a therapeutic mask for treating facial skin through the use of a hydrating mask and for aiding penetration of a topically applied treating composition.

2. Related Art

Some types of cosmetics products are intended to cover facial imperfections by application of substances which adhere to the surface of the skin. Other products are made to improve the appearance of facial imperfections by one of exfoliation, hydration and/or firming the skin. Typically, these products are lotions, creams or gels. While these products have met with some success, effectiveness of these products is limited in duration with typical visual effectiveness lasting less than an hour or several hours after application. These products have also proven to be less likely to penetrate sufficiently deep into the skin in a manner to provide a suitable lasting visual effect.

Adhesive patches are used to deliver pharmaceutical agents and cosmetic agents to the skin surfaces of humans. Many adhesive patches do not have an overall yield of product that is acceptable. The adhesive patches have relatively low water content and the adhesive tends to interfere with effectiveness.

It is desirable to provide a material onto the skin which will effectively improve the appearance of facial imperfections such as wrinkles or acne on the face. The material preferably can effectively exfoliate and hydrate the skin surface.

Honey has been noted to actually promote the healing process with no corresponding tissue damage. Honey is thought to provide anti-microbial properties as well as, non-peroxide activity, as well as its peroxide activity. The non-peroxide anti-bacterial activity of these honeys has been shown to inhibit the growth of various species of bacteria and limit the production of the undesirable bi-products of bacterial growth. Consequently, the use of honey on wounds is known within the prior art.

Although honey's use in treating wounds has met with some success, its fluidity does not lend itself to easy application. Some prior attempts exist to include honey in a therapeutic mask for purposes of treating wounds wherein the honey aids in absorbing exudates due to its hygroscopic nature.

Further aspects and advantages of the present invention will become apparent from the ensuing description which is given by way of example only.

### SUMMARY OF INVENTION

It is an object of the present invention to improve the condition of skin.

It is another object to improve the appearance of skin.

It is a further object to provide a method of treating skin.

It is still another object to provide a method of making a therapeutic mask for the treatment of skin.

It is yet another object to provide a therapeutic mask for treatment of skin.

It is a further object to aid in delivery of topically applied compositions.

According to one aspect of the present invention there is provided a therapeutic mask, wherein the mask includes a therapeutic composition including honey and a penetrating substance. Further, a moisture retaining substance, an emulsifier, a surfactant, a cellular repairing substance, an antioxidant, a keratin poietic, a humectant and a revitalizer, can also preferably be provided as part of the therapeutic composition. A dressing material having the therapeutic composition associated therewith is provided wherein the dressing material and therapeutic composition are characterized to be freeze dried to form the therapeutic mask.

Another aspect of the invention is directed to a method of manufacturing a therapeutic mask, the method including the steps of:

a) preparing a therapeutic composition including honey and at least one penetrating substance;

b) combining said therapeutic composition with a dressing material; and

c) freeze drying the combined dressing material and therapeutic composition to provide a therapeutic mask. The composition can include one or more of a moisture retaining substance, an emulsifier, a surfactant, a cellular repairing substance, an antioxidant, a keratin poietic, a humectant and a revitalizer.

Yet another embodiment of the invention provides for a method of treating skin, which includes the steps of providing (a) a therapeutic composition including honey and a penetrating substance, a dressing material having the therapeutic composition associated therewith, and wherein the dressing material and therapeutic composition are further characterized to be freeze dried to form the therapeutic mask and (b) rehyrdating the mask prior to use and (c) placing the therapeutic mask on mammalian skin for a period of time sufficient to permit the therapeutic composition to penetrate the skin. In one embodiment, an additional step is applying a dermal treatment composition prior to applying the therapeutic mask. The dermal treatment and/or composition can include a dermaceutical, CO₂ or laser. In another embodiment, the steps of removing the mask and then applying a second dermal treatment. The second dermal treatment can include at least one of an anesthetic or dermaceutical or laser, for example. Another step can be reapplying a therapeutic mask as described subsequent to applying the second dermal treatment.

The therapeutic mask can be formed with a relatively stretchable dressing having a plurality of open surfaces defined therein of a predetermined size and spatially positioned with respect to one another to accommodate the patient's eyes, nose and mouth. The therapeutic mask can further include retaining straps connectable to the elastic substrate and of a size and configuration to extend about the head of the patient for supporting the therapeutic mask in a relatively fixed position on the patient's face.

Other objects and advantages will be readily apparent to those skilled in the art upon viewing the drawings and reading the detailed description hereafter.

### BRIEF DESCRIPTION OF THE DRAWINGS

FIG. 1 is a perspective view of the present invention.

FIG. 2 depicts a microscopic view of untreated skin via hematoxylin and eosin.

FIG. 3 depicts a microscopic view of skin treated with the instant invention via hematoxylin and eosin.

FIG. 4 depicts a microscopic view of untreated skin via Giesma stain.

FIG. 5 depicts a microscopic view of skin treated with the instant invention via Giesma stain.

FIG. 6 depicts a microscopic view of untreated skin via Schorr stain.

FIG. 7 depicts a microscopic view of skin treated with the instant invention via Schorr stain.

FIG. 8 depicts a microscopic view of dermal plexus of untreated skin.

FIG. 9 depicts a microscopic view of dermal plexus of treated skin.

FIG. 10 depicts a microscopic view of treated dermis.

FIG. 11 depicts a microscopic view of treated dermis.

FIG. 12 depicts immunoenzynatic determination of dermal type III collagen of treated skin.

FIG. 13 depicts immunoenzynatic determination of dermal type III collagen of untreated skin.

FIG. 14 depicts a microscopic view of HSP 27 overexpression in treated skin.

FIG. 15 depicts a microscopic view of low HSP 27 expression in untreated skin.

FIG. 16 is a depiction of thin layer chromatography of calendula flavonoids.

FIG. 17 depicts a graph comparison of numbness with and without use of the instant invention.

FIG. 18 depicts a graph comparison of pain level with and without use of the instant invention.

### DETAILED DESCRIPTION OF THE INVENTION

Referring now to the drawings, the therapeutic mask of the invention is generally referred to by the numeral 10. There is also a method of manufacturing the therapeutic mask 10 and treating skin using the therapeutic mask 10. The therapeutic mask 10 includes a therapeutic composition 12 combined with a dressing material 14. It should be appreciated that variations to the ingredients of the therapeutic composition 12, the dressing material 14, and the methods of applying or impregnating the dressing material with the therapeutic composition 12 may be made and included within the scope of the present invention.

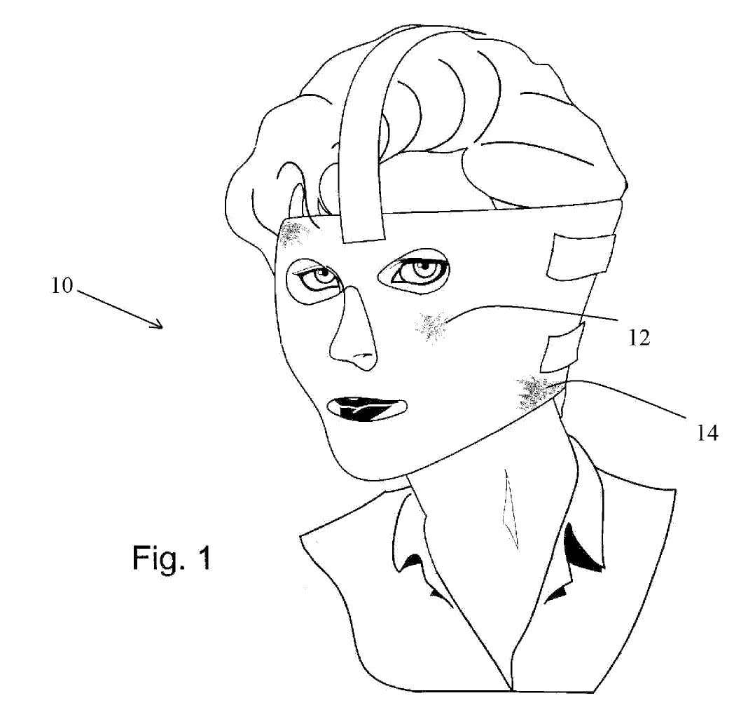

The dressing material 14 can vary in thickness depending on the material from which the therapeutic mask is made. A durable, loosely woven cotton or linen fabric can be used for the dressing material 14 and provided in the form of a patch or a full facial covering as seen in FIG. 1.

The therapeutic composition 12 can include honey and a penetrating substance. Additionally, the composition 12 can include one of a moisture retaining substance, an emulsifier, a surfactant, a cellular repairing substance, an antioxidant, a keratin poietic, a humectant and a revitalizer. The therapeutic composition 12 is combined with the dressing material 14 and freeze dried to provide the therapeutic mask 10.

Honey serves as a transfer vehicle believed to enable the delivery of the other chemical components in the mask 10. The following ingredients are added to honey vehicle and lyophilized in situ until moisture value reaches less than about 10%, and preferable about 6%. The dressing material 12 is activated when placed on wet skin or otherwise hydrated and placed in contact with skin.

An example of a suitable penetrating substance which can be employed is polyvinyl alcohol in the amounts of about 200 mg to 4000 mg (e.g., about 1200 mg in formula) as a plasticizing agent. This ingredient places a thin layer of plastic over the skin and enables the penetration as well as the mask 10 to be peeled off the skin. Film-forming agents can include a large group of ingredients that are typically found widely used in skin-care products, particularly moisturizers. These range from PVP to acrylates, acrylamides, and copolymers. When applied they leave a pliable, cohesive, and continuous covering over the hair or skin.

With the instant invention, the polyvinyl alcohol ingredient is important in holding the hydrating component, distilled water, while botanicals are activated from their freeze-dried state to rehydrate and revitalize the skin. This film produces excellent water-binding properties and leaves a smooth feel on skin.

A suitable emulsifying and emollient ingredient used skin-care products is LINOLEAMIDOPROPYL PG-DIMONIUM CHLORIDE PHOSPHATE 350 MG TO 130 MG (about 230 mg in formula). It is also an important ingredient in holding and activating the mask's botanical ingredients.

A surfactant ingredient can include propylene glycol 200 (PPG) in an amount of 400 MG TO 50 MG (about 200 mg in formula). A combination of glycerin and water, an organic humectant provides a clear, colorless liquid is a moisture-carrying vehicle in skin care. This combination aids in providing superb permeation through the skin and excellent humectant properties (softening and moisturizing the skin). OLEAMIDOPROPYL PG-DIMONIUM CHLORIDE 400 MG TO 50 MG (about 200 mg in formula) also can be provided as an emulsifier used to assist and balance the ingredients during the production of emollients.

Antioxidants and keratin poietic used in the invention can include vitamins C, A and E, e.g., Citric Acid, Vitamin E Acetate, Vitamin A Palmitate USP. Vitamin C is water soluble while A and E are fat soluble and the mixture of the non-mixable liquids is enabled by the surfactant PPG such that they continue to appear to be homogenized. Thus, the ingredients can be mixed together and applied to the dressing material 14 and lyophilized in situ until moisture value reaches 6% by freeze drying.

Active ingredients used in the invention include honey, e.g. about 350 mg (and range of about 450 mg to 10 mg) which contains about 75-80% sugar and the rest is a mixture of water and minerals like phosphorous, calcium, magnesium, some weak acids and enzymes. A skin repairer can include aloe vera and/or melaleuca alternafolia extracts. In the instant invention a range of about 450 mg to 10 mg, e.g., 350 mg in formula can be employed.

Vitamin A in an amount of range from about 125 to 5 mg can be employed and is considered a good antioxidant in some of its various forms, particularly as retinol and retinyl palmitate. Retinol is the entire vitamin A molecule, and it can be broken down into thousands of smaller components, of which one is retinoic acid (also called tretinoin, the active ingredient in Renova and Retin-A). Skin cells have a receptor site that is very accepting of retinoic acid. This relationship between retinoic acid and skin cells allows a type of communication in which the cell is told to function normally (that is, not like a damaged or older cell), and it can, to some extent, conform to that request. Retinol cannot communicate with a cell until it has been broken down into retinoic acid. It is thought retinol in skin-care products have problems in the stability in skin or in a skin-care product and whether it can be converted into retinoic acid after it is absorbed into the skin. Current thought is that retinol is a beneficial cell-communicating ingredient and an antioxidant but is very sensitive and packaging has been a key issue. Currently, containers that let in air (like jar packaging) or sunlight (clear containers) do not work. The instant invention provides an medium which maintains stability of this important Vitamin.

It is also noted that neither retinol nor retinoic acid appear to individually act to perform substantial benefit to skin on their own. For example, they don't replace the need for a well-formulated sunscreen, Alpha Hydroxy Acids (AHA) or Beta Hydroxy Acids (BHA) product. AHA and BHA have a long history of helping skin to function more normally by removing built-up layers of sun-damaged skin. Also, retinol is not the only ingredient desired in a moisturizer. AHA in various concentrations are used in chemical peels. AHA products sold to consumers have a concentration of less than 10%. Trained cosmetologists can use AHA products that have a concentration of 20% to 30%. These chemical peels give results that are similar to microdermabrasion—erasing fine lines and giving the skin a smoother appearance with 1 to 3 applications. However, these treatments must be repeated every 3 to 6 months to maintain this skin appearance. Doctors can use AHA products that have a concentration of 50% to 70%. Skin needs a combination of ingredients to function optimally, including cell-communicating ingredients (of which retinol is one), antioxidants (to reduce free-radical damage), and substances that mimic skin structure. Together, all these various ingredients and elements combine to create a powerful part of any skin-care routine. The use of BHA can increase sun sensitivity by 50% causing an interesting dilemma. It appears that BHA may be able to reverse some of the damage caused by photoaging, but at the same time it makes the skin more susceptible to photoaging. It is clear that anyone using BHA must use a good sunscreen that contains UVA and UVB protection.

Vitamin C can be used in an amount range of about 2500 to 10 mg and is considered a potent antioxidant for skin. Topical vitamin C does not absorb or block harmful ultraviolet radiation like a sunscreen. Instead, it augments the skin's ability to neutralize reactive oxygen singlets [free-radical damage] that are created by the ultraviolet radiation, thereby preventing photodamage to the skin. It becomes an integral part of the skin and remains unaffected by bathing, exercise, clothing, or makeup. Topical vitamin C is an important adjunct to the use of sunscreens, an adjunctive treatment to lessen erythema [redness] in skin resurfacing, a helpful adjunct or an alternative to Retin-A in the treatment of fine wrinkles, and a stimulant to wound healing.

Vitamin E can be employed in an amount range from about 300 mg to 10 mg and is considered an antioxidant superstar. Vitamin E is a lipid-soluble vitamin (meaning it likes fat better than water) that has eight different forms, of which some are known for being excellent antioxidants when applied topically to skin, particularly alpha tocopherol and the tocotrienols. Acetate form (tocopherol acetate) is also bioavailable and protective for skin and tocopherol sorbate provides significant antioxidant protection against ultraviolet radiation-induced oxidative damage.

A suitable moisture retention ingredient can include Sodium-2-pyrrolidone carboxylate (PCA) in an amount of range of about 450 mg to 10 mg. Sodium PCA is hygroscopic, attracting moisture from the air. It is used as a moisturizer (humectant) for hair and skin care products.

It is a stronger hydrating agent than the traditional compounds used for this purpose, such as glycerine, propylene glycol, or sorbitol. One of the primary elements in keeping skin healthy is making sure the structure of the epidermis (outer layer of skin) is intact. That structure is defined and created by skin cells that are held together by the intercellular matrix. The intercellular matrix is the “glue” or “mortar” between skin cells that keep them together. Sodium PCA helps prevent individual skin cells from losing water and creates the smooth, non-flaky appearance of healthy, intact skin. The components that do this are often called natural moisturizing factors (NMFs) or ingredients that mimic the structure and function of healthy skin. While the oil and fat components of skin prevent evaporation and provide lubrication to the surface of skin, it is actually the intercellular matrix along with the skin's lipid content that gives skin a good deal of its surface texture and feel.

The intercellular matrix is the skin's first line of defense against water loss. When the lipid and NMF content of skin is reduced, we experience surface roughness, flaking, fine lines, and a tight, uncomfortable feeling. The longer the skin's surface layer (stratum corneum) is impaired, the less effective the skin's intercellular matrix becomes. The skin's healing process is impaired. NMFs make up an expansive group of ingredients that include amino acids, ceramides, hyaluronic acid, cholesterol, fatty acids, triglycerides, phospholipids, glycosphingolipids, urea, linoleic acid, glycosaminoglycans, glycerin, mucopolysaccharide, and sodium PCA (pyrrolidone carboxylic acid). Ingredients that mimic the lipid content of skin are apricot oil, canola oil, coconut oil, corn oil, jojoba oil, jojoba wax, lanolin, lecithin, olive oil, safflower oil, sesame oil, shea butter, soybean oil, squalane, and sweet almond oil, which can all be extremely helpful for making dry skin look and feel better.

All of the skin's supporting NMFs and lipids are present in the intercellular structure of the epidermis, both between skin cells and in the lipid content on the surface of skin. When any of these ingredients are used in skin-care products, they appear to help stabilize and maintain this complex intercellular-skin matrix. Although none of these very good NMFs and lipids can permanently affect or change skin, they are great at temporarily keeping depleted skin from feeling dry and uncomfortable. More important, all of these ingredients, and many more, can help support the intercellular area of the skin by keeping it intact. This support helps prevent surface irritation from penetrating deeper into the skin, works to keep bacteria out, and aids the skin's immune/healing system. NMF allows the skin to do its job of repairing and regenerating itself without the impedances brought on when skin is suffering from dryness and excess irritation.

The humectants and revitalizers of the instant invention can include a variety of botanical compounds which contain natural substances such as alpha-hydroxy acid, which aid the mask's exfoliation properties. For example, such humectants and revitalizers include botanicals in the range from about 350 to 10 mg. Such botanical includes: burdock root which is thought to be effective as an anti-inflammatory agent and antioxidant; calendula extract derived from the plant commonly known as pot marigold which may have antibacterial and antioxidant properties for skin; comfrey extract is thought to have an alkaloid content and makes it a potential skin irritant; cucumber extract is thought to provide anti-inflammatory or soothing properties; ephedra sinica extract is a Chinese herb also known as Ma huang and has a high tannin and volatile oil content and toxic properties; elderberry is known for potent antioxidant properties; germanium which is sometimes is identified as vitamin O; potentilla erecta root extract which is thought to have anti-inflammatory properties; plantain which is thought to clear mucous from the body and to neutralize poisons; and tormentil extract acts as a styptic to cuts, wounds, etc.

The therapeutic composition 14 may be fully impregnated and/or coated onto the dressing material 12.

### EXAMPLE

The mask 10 can include a net-like occlusive scrim on one side with a mixture of various components for example polyvinyl alcohol, linoleanidopropyl D-G diammonium chloride, Vitamins C, A and E, sodium JPCA and botanical extracts: aloe vera, common comfrey, geranium, calendula, elderberry flower, burdock, ephedra, cucumber, plantain, and tormentil, one or more of which is in a honey-based vehicle. All compounds were mixed in proper proportions in a reactor at a controlled temperature, then quick frozen at −35° C. and later stabilized at 10° C. The mixture is then applied to the occlusive net-like scrim film and lyophilized until the moisture content reaches approximately 6%. When ready for facial application, the mask 10 is activated with distilled water and applied directly the skin.

The following experiments were preformed with the aim of determining the mode of action and the effects on the cutaneous surface.

Materials and Methods Patients: 24 female patients between the ages of 38 and 64 with breast carcinoma, who were scheduled to undergo a mastectomy for their disease volunteered and were selected for the study. All were informed about the nature of the tests to be done and each patient gave their written consent. None of the test subjects had any dermatological condition which would prevent participation. The patients were divided into four groups of six each.

Prior to mastectomy a piece or mask material for testing (4×3 cm) was placed under the areola, activated with distilled water, and left in place for 45 minutes. Group A received the test mask material immediately before surgery, during induction of anesthesia; Group B received the test mask material one hour before surgery; Group C received the mask three hours before surgery; and Group D six hours before surgery. The average estimated time of the surgical procedure was approximately 45 minutes.

TABLE I
Surface dermal plexus vascular diameters (mm) for treated and untreated skin samples.
were performed per tissue sample (U: untreated; T: treated; Group A: samples obtained
removal; Group B: samples obtained 1 hour after material removal; Group C: samples
material removal; Group D: samples obtained 6 hours after material removal; VOL:
deviation; Max.: maximum; Min.:

| | MEASUREMENT | | | | | | | | | RANGE | |
|---|---|---|---|---|---|---|---|---|---|---|---|
| GROUP | VOL. | 1 | 2 | 3 | 4 | 5 | Sum | Mean | S.D. | Max. | Min. |
| A | 1 T | 0.11 | 0.07 | 0.07 | 0.06 | 0.10 | 0.41 | 0.08 | 0.02 | 0.11 | 0.06 |
| | 1 U | 0.05 | 0.06 | 0.06 | 0.06 | 0.06 | 0.28 | 0.06 | 0.00 | 0.06 | 0.05 |
| | 2 T | 0.10 | 0.07 | 0.09 | 0.07 | 0.10 | 0.43 | 0.09 | 0.01 | 0.10 | 0.07 |
| | 2 U | 0.05 | 0.06 | 0.05 | 0.05 | 0.06 | 0.27 | 0.05 | 0.00 | 0.06 | 0.05 |
| | 3 T | 0.07 | 0.08 | 0.11 | 0.07 | 0.06 | 0.39 | 0.08 | 0.02 | 0.11 | 0.06 |
| | 3 U | 0.06 | 0.05 | 0.06 | 0.06 | 0.04 | 0.27 | 0.05 | 0.01 | 0.06 | 0.04 |
| | 4 T | 0.08 | 0.10 | 0.09 | 0.08 | 0.07 | 0.42 | 0.08 | 0.02 | 0.10 | 0.07 |
| | 4 U | 0.05 | 0.05 | 0.06 | 0.06 | 0.05 | 0.27 | 0.05 | 0.00 | 0.06 | 0.05 |
| | 5 T | 0.09 | 0.09 | 0.07 | 0.10 | 0.07 | 0.42 | 0.08 | 0.02 | 0.10 | 0.07 |
| | 5 U | 0.06 | 0.06 | 0.05 | 0.06 | 0.05 | 0.28 | 0.06 | 0.00 | 0.06 | 0.05 |
| | 6 T | 0.10 | 0.09 | 0.09 | 0.07 | 0.10 | 0.45 | 0.09 | 0.02 | 0.10 | 0.07 |
| | 6 U | 0.05 | 0.06 | 0.06 | 0.05 | 0.06 | 0.28 | 0.06 | 0.00 | 0.06 | 0.05 |
| B | 7 T | 0.07 | 0.07 | 0.08 | 0.08 | 0.08 | 0.38 | 0.08 | 0.01 | 0.08 | 0.07 |
| | 7 U | 0.06 | 0.04 | 0.05 | 0.05 | 0.06 | 0.26 | 0.05 | 0.01 | 0.06 | 0.04 |
| | 8 T | 0.06 | 0.08 | 0.08 | 0.06 | 0.10 | 0.38 | 0.08 | 0.02 | 0.10 | 0.06 |
| | 8 U | 0.05 | 0.05 | 0.06 | 0.06 | 0.06 | 0.28 | 0.06 | 0.00 | 0.06 | 0.05 |
| | 9 T | 0.07 | 0.07 | 0.06 | 0.08 | 0.07 | 0.35 | 0.07 | 0.01 | 0.08 | 0.06 |
| | 9 U | 0.06 | 0.04 | 0.05 | 0.05 | 0.05 | 0.25 | 0.05 | 0.01 | 0.06 | 0.04 |
| | 10 T | 0.09 | 0.06 | 0.06 | 0.07 | 0.08 | 0.36 | 0.07 | 0.02 | 0.09 | 0.06 |
| | 10 U | 0.05 | 0.05 | 0.06 | 0.06 | 0.05 | 0.27 | 0.05 | 0.00 | 0.06 | 0.05 |
| | 11 T | 0.08 | 0.07 | 0.07 | 0.08 | 0.08 | 0.36 | 0.07 | 0.01 | 0.08 | 0.07 |
| | 11 U | 0.06 | 0.06 | 0.05 | 0.05 | 0.05 | 0.26 | 0.05 | 0.00 | 0.06 | 0.05 |
| | 12 T | 0.10 | 0.07 | 0.09 | 0.06 | 0.06 | 0.38 | 0.08 | 0.02 | 0.10 | 0.06 |
| | 12 U | 0.05 | 0.05 | 0.07 | 0.06 | 0.06 | 0.29 | 0.06 | 0.01 | 0.07 | 0.05 |
| C | 13 T | 0.06 | 0.07 | 0.06 | 0.08 | 0.05 | 0.32 | 0.06 | 0.02 | 0.08 | 0.05 |
| | 13 U | 0.05 | 0.05 | 0.06 | 0.06 | 0.05 | 0.27 | 0.05 | 0.00 | 0.06 | 0.05 |
| | 14 T | 0.07 | 0.05 | 0.06 | 0.06 | 0.07 | 0.31 | 0.06 | 0.01 | 0.07 | 0.05 |
| | 14 U | 0.04 | 0.06 | 0.06 | 0.05 | 0.05 | 0.26 | 0.05 | 0.01 | 0.06 | 0.04 |
| | 15 T | 0.06 | 0.06 | 0.07 | 0.07 | 0.07 | 0.33 | 0.07 | 0.00 | 0.07 | 0.06 |
| | 15 U | 0.05 | 0.06 | 0.06 | 0.06 | 0.06 | 0.29 | 0.06 | 0.00 | 0.06 | 0.05 |
| | 16 T | 0.06 | 0.06 | 0.07 | 0.06 | .06 | 0.31 | 0.06 | 0.00 | 0.07 | 0.06 |
| | 16 U | 0.07 | 0.05 | 0.06 | 0.06 | 0.04 | 0.28 | 0.06 | 0.02 | 0.07 | 0.04 |
| | 17 T | 0.06 | 0.06 | 0.06 | 0.06 | 0.05 | 0.29 | 0.06 | 0.00 | 0.06 | 0.05 |
| | 17 U | 0.06 | 0.05 | 0.05 | 0.06 | 0.05 | 0.26 | 0.05 | 0.00 | 0.06 | 0.05 |
| | 18 T | 0.07 | 0.08 | 0.07 | 0.06 | 0.06 | 0.34 | 0.07 | 0.01 | 0.08 | 0.06 |
| | 18 U | 0.06 | 0.06 | 0.05 | 0.06 | 0.05 | 0.28 | 0.06 | 0.00 | 0.06 | 0.05 |
| D | 19 T | 0.06 | 0.07 | 0.05 | 0.06 | 0.06 | 0.30 | 0.06 | 0.01 | 0.07 | 0.05 |
| | 19 U | 0.06 | 0.05 | 0.06 | 0.06 | 0.06 | 0.29 | 0.06 | 0.00 | 0.06 | 0.05 |
| | 20 T | 0.04 | 0.06 | 0.05 | 0.06 | 0.04 | 0.25 | 0.05 | 0.01 | 0.06 | 0.04 |
| | 20 U | 0.06 | 0.05 | 0.05 | 0.05 | 0.05 | 0.26 | 0.05 | 0.00 | 0.06 | 0.05 |
| | 21 T | 0.07 | 0.04 | 0.05 | 0.06 | 0.06 | 0.28 | 0.06 | 0.01 | 0.07 | 0.04 |
| | 21 U | 0.06 | 0.05 | 0.06 | 0.06 | 0.07 | 0.30 | 0.06 | 0.01 | 0.07 | 0.05 |
| | 22 T | 0.07 | 0.06 | 0.07 | 0.06 | 0.07 | 0.33 | 0.07 | 0.00 | 0.07 | 0.06 |
| | 22 U | 0.06 | 0.05 | 0.07 | 0.05 | 0.06 | 0.29 | 0.06 | 0.01 | 0.07 | 0.05 |
| | 23 T | 0.05 | 0.05 | 0.05 | 0.06 | 0.04 | 0.25 | 0.05 | 0.01 | 0.06 | 0.04 |
| | 23 U | 0.06 | 0.06 | 0.05 | 0.05 | 0.04 | 0.26 | 0.05 | 0.01 | 0.06 | 0.04 |
| | 24 T | 0.05 | 0.05 | 0.06 | 0.05 | 0.06 | 0.27 | 0.05 | 0.00 | 0.06 | 0.05 |
| | 24 U | 0.05 | 0.06 | 0.05 | 0.05 | 0.05 | 0.26 | 0.05 | 0.00 | 0.06 | 0.05 |

Immediately upon completion of the resection of each breast, the skin samples of the treated area was extracted along with skin samples of the same size from an adjacent untreated area which acted as an untreated control. Both treated and untreated skin samples were cut into fragments for light and electron microscopy studies. Samples of skin from each group were frozen in carbon dioxide for cutaneous absorption testing. The remaining test material was used to determine moisture absorption rates.

A. Determination of Moisture Content

Each sample of treated and untreated skin was weighed separately and then placed in an oven at 100° C. for thirty minutes. The samples were again weighed and moisture content determined by measuring the evaporated water with the following formula:

(X1−X2)/X1×100

where X1 is the weight of the sample before the evaporation and X2 is the weight of the sample alter heat exposure.

B. Light Microscopy Examinations

Each sample was fixed in a formal in buffered solution pH 6.8 for 20 hours, the samples were then dehydrated by passage into increasing graduated alcohol solutions, embedded in paraplast, and cut into 5 m thickness and stained with routine hematoxylin and eosin, Giemsa stain, PAS, Schorr stain and methylene blue.

The stratum corneum as well as the whole epidermal thickness was then measured, together with the luminal diameters of the dermal plexus capillaries, using an image processing computer with a morphometry program and Quantimet 500-Plus Leica camera. Vascular surfaces were measured in a similar manner only if the vessels were cut perpendicular to their axis. Vessels with ovoid or elongate shapes were not measured.

C. Ultrasound Studies

One sample of treated, as well as untreated skin, were embedded for a period of twenty-four hours in 2% glutaraldehyde solution and post-fixed with a 1% osmium tetroxide solution. They were then dehydrated in increasingly graduated alcohol, including araldite. Next they were cut with an ultramicrotome, contrasted with lead citrate and uranyl acetate, and examined with a Phillips EM200 transmission electron microscope.

D. Immunohistochemical Studies

Sections of both treated and untreated skin processed for light microscopy were deparaffinated, rehydrated and incubated for 30 minutes with commercial monoclonal antibodies heat shock proteins (HSPs) of different molecular weights:

1. HSP27—Biogenex

2. HSP70, HSP84 and HSP 100 Affinity Bioreagents, Inc.

3. Type III collagen antibodies Biogenex

Reactions were developed with commercial kits containing alkaline phosphate for LISP—APAAP Quick Staining Kit and with a peroxidase anti-peroxide system—PAP Kit all from Dako.

E. Absorption Studies

In order to assess the absorption capacity of the skin stimulated by the test mask material the presence of one of the botanical components was measured, at different depths of the stratum cutaneous, using the following method: sections of skin were cut into 100 m thickness with a cryostat out of a frozen a section. Cuts were parallel to the cutaneous surface; each section was numbered successively according to its depth of cut.

Every specimen underwent a dialysis process through Vickin membranes in distilled water for 24 hours at 4° C. The dialysis liquid was concentrated in Merck Extrelut filling columns and eluted with 40 ml of dichloromethane and 40 ml ethyl ether. The eluate was dried and rehydrated with absolute alcohol. The final product was inoculated in C3F254 Merck silica gel plates for thin layer chromatography (TLC) with methanol: ammonia mobile phase (100:1.5). The development was achieved by exposing the plate to ultraviolet light with wavelengths of 254 to 366 nanometers. This method was chosen after several tests were designed to determine which botanical extract would best fit the analytical method used. As a result the botanical calendula extract was selected. This extract is rich in flavonoids, especially the flavonoid fraction methylated aglycons.

Results

A. Assessment of the moisture content Results in Table II demonstrate that after 45 to 60 minutes the mean difference in moisture was 30.1873% between treated and untreated skin samples, which gradually decreases in value with longer time intervals of application, with the lowest values in samples six hours after mask material removal.

B. Light Microscopy Studies

For the group of patients in which the study was performed immediately after the mask material removal, evident morphologic changes were observed in all cases in the epidermal stratum corneum. The epidermal stratum corneum showed more thickening in treated skin than did the untreated skin. As the interval between mask material removal and sampling grew larger, stratum corneum thickness was reduced, tending to resemble the controls.

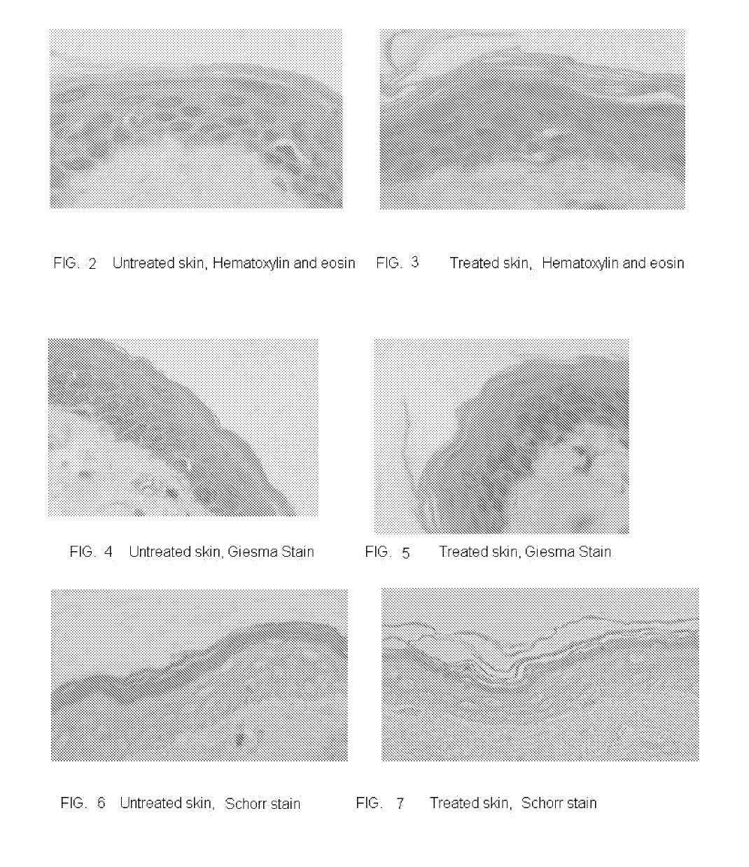

Morphologically, the change in stratum corneum was evidenced by a gap between the layers, whereas control skin samples showed compact corneum layers. The same was true of the dermal papilae in the treated skin; they showed an enlargement of their bases (see FIGS. 2, 3, 4, 5, 6, and 7). In the groups of patients with the greatest interval between mask material application and removal, these changes were less evident.

TABLE II
Moisture rates of treated skin vs. untreated skin, at different times after material
removal. (U: untreated; T: treated; X1: weight in grams before evaporated; Group A:
samples obtained immediately after material removal; Group B: samples obtained
1 hour after material removal; Group C: samples obtained immediately 3 hours
after material removal; Group D: samples obtained 6 hours 6 hours after
material removal.

| GRUP | SAMPLE | X1 | X2 | Moisture (%) | Difference T − U (%) | Mean (%) |
|---|---|---|---|---|---|---|
| A | 1 U | 69.2400 | 67.6693 | 2.26848 | | |
| | 1 T | 28.7910 | 27.9366 | 2.96759 | 30.818% | |
| | 2 U | 34.2510 | 33.4638 | 2.29832 | | |
| | 2 T | 38.0090 | 36.8795 | 2.97166 | 29.297% | |
| | 3 U | 47.0150 | 45.9476 | 2.27033 | | |
| | 3 T | 49.8266 | 48.3413 | 2.98093 | 31.230% | |
| | 4 U | 26.8114 | 26.1972 | 2.29081 | | |
| | 4 T | 34.0050 | 32.9980 | 2.96132 | 29.230% | |
| | 5 U | 52.5963 | 51.525 | 2.22235 | | |
| | 5 T | 50.4250 | 48.9575 | 2.91026 | 30.954% | |
| | 6 U | 41.1126 | 40.1862 | 2.25325 | | |
| | 6 T | 39.9902 | 38.8228 | 2.91921 | 29.555% | 30.1873% |
| B | 7 U | 49.0455 | 47.9460 | 2.29325 | | |
| | 7 T | 53.3911 | 51.9302 | 2.81319 | 22.673% | |
| | 8 U | 61.4834 | 60.1227 | 2.26316 | | |
| | 8 T | 56.5042 | 54.9527 | 2.82325 | 24.748% | |
| | 9 U | 44.0725 | 43.1104 | 2.23179 | | |
| | 9 T | 52.4364 | 51.0026 | 2.81127 | 25.965% | |
| | 10 U | 28.4220 | 27.8039 | 2.22231 | | |
| | 10 T | 30.1325 | 29.3020 | 2.83428 | 27.502% | |
| | 11 U | 33.1629 | 32.4289 | 2.26336 | | |
| | 11 T | 39.3624 | 36.1122 | 2.90002 | 28.129% | |
| | 12 U | 51.0445 | 49.9260 | 2.24038 | | |
| | 12 T | 52.8135 | 51.3254 | 2.89932 | 29.416% | 26.4055% |
| C | 13 U | 44.3105 | 43.3251 | 2.27451 | | |
| | 13 T | 42.4091 | 41.3262 | 2.62043 | 15.209% | |
| | 14 U | 50.0341 | 48.9265 | 2.26388 | | |
| | 14 T | 49.0470 | 47.8321 | 2.53992 | 12.194% | |
| | 15 U | 61.3695 | 60.0232 | 2.22430 | | |
| | 15 T | 63.5528 | 62.0034 | 2.49896 | 10.592% | |
| | 16 U | 29.6925 | 29.0439 | 2.23332 | | |
| | 16 T | 28.5751 | 27.8672 | 2.54028 | 13.745% | |
| | 17 U | 30.1085 | 29.4446 | 2.25480 | | |
| | 17 T | 31.2193 | 30.4251 | 2.61036 | 15.769% | |
| | 18 U | 42.9726 | 42.0380 | 2.22315 | | |
| | 18 T | 49.0546 | 47.8352 | 2.54922 | 14.667% | 13.6960% |
| D | 19 U | 54.2025 | 53.0231 | 2.22438 | | |
| | 19 T | 54.1537 | 52.9280 | 2.31589 | 4.119% | |
| | 20 U | 43.2831 | 42.3265 | 2.26006 | | |
| | 20 T | 46.4729 | 45.3880 | 2.39032 | 5.763% | |
| | 21 U | 30.0833 | 29.4267 | 2.23723 | | |
| | 21 T | 28.0377 | 27.3990 | 2.33121 | 4.201% | |
| | 22 U | 61.3610 | 60.0340 | 2.21039 | | |
| | 22 T | 63.2612 | 61.8262 | 2.32105 | 5.006% | |
| | 23 U | 34.0944 | 33.3428 | 2.25425 | | |
| | 23 T | 33.2511 | 32.4760 | 2.38684 | 5.882% | |
| | 24 U | 48.4446 | 47.3828 | 2.24080 | | |
| | 24 T | 47.1677 | 46.1114 | 2.29072 | 2.228% | 4.5323% |

TABLE III
Corneum stratum thickness in (mm) for treated and untreated skin. Five
measurements were performed per sample (U: untreated; Group A:
samples obtained immediately after material removal; Group B:
samples obtained 1 hour after material removal; Group C: samples
obtained 3 hours after material removal; Group D: samples Obtained
6 hours after material removal; VOL: Volunteer; SD: standard
deviation; Max.: maximum; Min.: Minimum)

| | | MEASUREMENT | | | | | | | | RANGE | |
|---|---|---|---|---|---|---|---|---|---|---|---|
| GRUP | VOL. | 1 | 2 | 3 | 4 | 5 | Sum | Mean | S.D. | Max. | Min. |
| A | 1 T | 0.11 | 0.13 | 0.11 | 0.14 | 0.10 | 0.59 | 0.12 | 0.01 | 0.14 | 0.10 |
| | 1 U | 0.06 | 0.06 | 0.06 | 0.07 | 0.09 | 0.34 | 0.07 | 0.02 | 0.09 | 0.06 |
| | 2 T | 0.14 | 0.11 | 0.13 | 0.16 | 0.11 | 0.65 | 0.13 | 0.02 | 0.16 | 0.11 |
| | 2 U | 0.06 | 0.07 | 0.06 | 0.07 | 0.07 | 0.33 | 0.07 | 0.01 | 0.07 | 0.06 |
| | 3 T | 0.11 | 0.13 | 0.15 | 0.10 | 0.11 | 0.60 | 0.12 | 0.02 | 0.15 | 0.10 |
| | 3 U | 0.06 | 0.07 | 0.08 | 0.06 | 0.07 | 0.34 | 0.07 | 0.01 | 0.08 | 0.06 |
| | 4 T | 0.10 | 0.11 | 0.14 | 0.13 | 0.12 | 0.60 | 0.12 | 0.01 | 0.14 | 0.10 |
| | 4 U | 0.05 | 0.06 | 0.06 | 0.07 | 0.07 | 0.31 | 0.06 | 0.01 | 0.07 | 0.05 |
| | 5 T | 0.12 | 0.11 | 0.13 | 0.10 | 0.12 | 0.58 | 0.12 | 0.01 | 0.13 | 0.10 |
| | 5 U | 0.06 | 0.05 | 0.06 | 0.06 | 0.05 | 0.28 | 0.06 | 0.01 | 0.06 | 0.05 |
| | 6 T | 0.11 | 0.12 | 0.12 | 0.13 | 0.13 | 0.61 | 0.12 | 0.01 | 0.13 | 0.11 |
| | 6 U | 0.06 | 0.0 | 0.07 | 0.05 | 0.05 | 0.30 | 0.06 | 0.01 | 0.07 | 0.05 |
| B | 7 T | 0.11 | 0.10 | 0.11 | 0.11 | 0.10 | 0.53 | 0.11 | 0.01 | 0.11 | 0.10 |
| | 7 U | 0.06 | 0.05 | 0.06 | 0.06 | 0.05 | 0.28 | 0.06 | 0.01 | 0.06 | 0.05 |
| | 8 T | 0.11 | 0.11 | 0.10 | 0.09 | 0.10 | 0.51 | 0.10 | 0.01 | 0.11 | 0.09 |
| | 8 U | 0.06 | 0.07 | 0.06 | 0.06 | 0.06 | 0.31 | 0.06 | 0.01 | 0.07 | 0.06 |
| | 9 T | 0.10 | 0.11 | 0.10 | 0.10 | 0.11 | 0.52 | 0.10 | 0.01 | 0.11 | 0.10 |
| | 9 U | 0.07 | 0.07 | 0.07 | 0.08 | 0.08 | 0.33 | 0.07 | 0.01 | 0.07 | 0.06 |
| | 10 T | 0.10 | 0.10 | 0.09 | 0.09 | 0.10 | 0.48 | 0.10 | 0.01 | 0.10 | 0.09 |
| | 10 U | 0.07 | 0.06 | 0.06 | 0.06 | 0.06 | 0.31 | 0.06 | 0.01 | 0.07 | 0.06 |
| | 11 T | 0.11 | 0.09 | 0.11 | 0.10 | 0.11 | 0.52 | 0.10 | 0.01 | 0.11 | 0.09 |
| | 11 U | 0.07 | 0.07 | 0.06 | 0.07 | 0.06 | 0.33 | 0.07 | 0.01 | 0.07 | 0.06 |
| | 12 T | 0.11 | 0.11 | 0.10 | 0.10 | 0.09 | 0.51 | 0.10 | 0.01 | 0.11 | 0.09 |
| | 12 U | 0.06 | 0.06 | 0.06 | 0.05 | 0.06 | 0.28 | 0.06 | 0.01 | 0.06 | 0.05 |
| C | 13 T | 0.09 | 0.09 | 0.08 | 0.10 | 0.09 | 0.45 | 0.09 | 0.01 | 0.10 | 0.08 |
| | 13 U | 0.06 | 0.07 | 0.06 | 0.05 | 0.06 | 0.30 | 0.06 | 0.01 | 0.07 | 0.05 |
| | 14 T | 0.10 | 0.10 | 0.09 | 0.09 | 0.09 | 0.47 | 0.09 | 0.01 | 0.10 | 0.09 |
| | 14 U | 0.06 | 0.06 | 0.07 | 0.06 | 0.06 | 0.31 | 0.06 | 0.01 | 0.07 | 0.06 |
| | 15 T | 0.10 | 0.08 | 0.08 | 0.10 | 0.09 | 0.45 | 0.09 | 0.01 | 0.10 | 0.08 |
| | 15 U | 0.07 | 0.06 | 0.06 | 0.07 | 0.05 | 0.31 | 0.05 | 0.01 | 0.07 | 0.05 |
| | 16 T | 0.09 | 0.08 | 0.09 | 0.09 | 0.08 | 0.43 | 0.09 | 0.01 | 0.09 | 0.08 |
| | 16 U | 0.05 | 0.05 | 0.06 | 0.06 | 0.06 | 0.28 | 0.06 | 0.01 | 0.06 | 0.05 |
| | 17 T | 0.09 | 0.09 | 0.08 | 0.09 | 0.09 | 0.44 | 0.09 | 0.01 | 0.09 | 0.08 |
| | 17 U | 0.06 | 0.06 | 0.07 | 0.05 | 0.06 | 0.30 | 0.05 | 0.01 | 0.07 | 0.05 |
| | 18 T | 0.09 | 0.09 | 0.07 | 0.08 | 0.10 | 0.43 | 0.09 | 0.02 | 0.10 | 0.07 |
| | 18 U | 0.07 | 0.06 | 0.06 | 0.07 | 0.07 | 0.33 | 0.07 | 0.01 | 0.07 | 0.06 |
| D | 19 T | 0.06 | 0.07 | 0.06 | 0.05 | 0.06 | 0.30 | 0.06 | 0.01 | 0.07 | 0.05 |
| | 19 U | 0.06 | 0.05 | 0.05 | 0.08 | 0.06 | 0.28 | 0.06 | 0.01 | 0.06 | 0.05 |
| | 20 T | 0.05 | 0.07 | 0.07 | 0.06 | 0.06 | 0.31 | 0.06 | 0.01 | 0.07 | 0.05 |
| | 20 U | 0.06 | 0.05 | 0.04 | 0.06 | 0.05 | 0.26 | 0.05 | 0.01 | 0.06 | 0.04 |
| | 21 T | 0.07 | 0.06 | 0.06 | 0.07 | 0.07 | 0.33 | 0.07 | 0.01 | 0.07 | 0.06 |
| | 21 U | 0.06 | 0.06 | 0.06 | 0.06 | 0.06 | 0.30 | 0.06 | 0.00 | 0.06 | 0.06 |
| | 22 T | 0.06 | 0.06 | 0.07 | 0.06 | 0.06 | 0.31 | 0.06 | 0.01 | 0.07 | 0.06 |
| | 22 U | 0.06 | 0.05 | 0.05 | 0.06 | 0.06 | 0.28 | 0.06 | 0.01 | 0.06 | 0.05 |
| | 23 T | 0.05 | 0.07 | 0.06 | 0.06 | 0.07 | 0.31 | 0.06 | 0.01 | 0.07 | 0.05 |
| | 23 U | 0.06 | 0.06 | 0.05 | 0.06 | 0.06 | 0.29 | 0.06 | 0.01 | 0.06 | 0.05 |
| | 24 T | 0.06 | 0.06 | 0.07 | 0.07 | 0.06 | 0.32 | 0.06 | 0.01 | 0.07 | 0.06 |
| | 24 U | 0.07 | 0.06 | 0.06 | 0.06 | 0.07 | 0.32 | 0.06 | 0.01 | 0.07 | 0.06 |

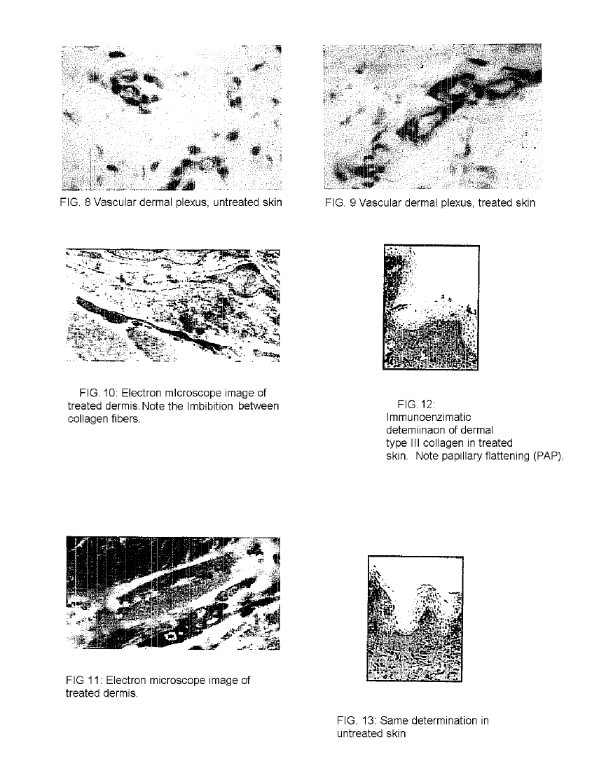

A marked difference in vascular diameter measurements between treated skin and controls is evident as seen in Table IV and FIGS. 8 and 9. Vascular diameters progressively decreased between treated skin and untreated skin and became no longer evident, six hours after material removal. Vascular surface values correlated exactly with vascular diameter values obtained.

TABLE IV
Surface dermal plexus vascular diameters (mm) for treated skin. Five
measurements were performed per sample (U: untreated; T: treated;
Group A: obtained immediately after material removal; Group B;
samples obtained 1 hour after material removal; Group C: samples
obtained after 3 hours after material removal; Group D: samples
obtained 6 hours after material removal; VOL: Volunteer; SD: standard
deviation; Max.: maximum; Min.: Minimum)

| | | MEASUREMENT | | | | | | | | RANGE | |
|---|---|---|---|---|---|---|---|---|---|---|---|
| GRUP | VOL. | 1 | 2 | 3 | 4 | 5 | Sum | Mean | S.D. | Max. | Min. |
| A | 1 T | 0.11 | 0.13 | 0.11 | 0.14 | 0.10 | 0.59 | 0.12 | 0.01 | 0.14 | 0.10 |
| | 1 U | 0.06 | 0.06 | 0.06 | 0.07 | 0.09 | 0.34 | 0.07 | 0.02 | 0.09 | 0.06 |
| | 2 T | 0.14 | 0.11 | 0.13 | 0.16 | 0.11 | 0.65 | 0.13 | 0.02 | 0.16 | 0.11 |
| | 2 U | 0.06 | 0.07 | 0.06 | 0.07 | 0.07 | 0.33 | 0.07 | 0.01 | 0.07 | 0.06 |
| | 3 T | 0.11 | 0.13 | 0.15 | 0.10 | 0.11 | 0.60 | 0.12 | 0.02 | 0.15 | 0.10 |
| | 3 U | 0.06 | 0.07 | 0.08 | 0.06 | 0.07 | 0.34 | 0.07 | 0.01 | 0.08 | 0.06 |
| | 4 T | 0.16 | 0.11 | 0.14 | 0.13 | 0.12 | 0.60 | 0.12 | 0.01 | 0.14 | 0.10 |
| | 4 U | 0.05 | 0.06 | 0.08 | 0.07 | 0.07 | 0.31 | 0.06 | 0.01 | 0.07 | 0.05 |
| | 5 T | 0.12 | 0.11 | 0.13 | 0.10 | 0.12 | 0.58 | 0.12 | 0.01 | 0.13 | 0.10 |
| | 5 U | 0.06 | 0.05 | 0.06 | 0.06 | 0.05 | 0.28 | 0.06 | 0.01 | 0.06 | 0.05 |
| | 6 T | 0.11 | 0.12 | 0.12 | 0.13 | 0.13 | 0.61 | 0.12 | 0.01 | 0.13 | 0.11 |
| | 6 U | 0.06 | 0.0 | 0.07 | 0.05 | 0.06 | 0.30 | 0.06 | 0.01 | 0.07 | 0.05 |
| B | 7 T | 0.11 | 0.10 | 0.11 | 0.11 | 0.10 | 0.53 | 0.11 | 0.01 | 0.11 | 0.10 |
| | 7 U | 0.06 | 0.05 | 0.06 | 0.06 | 0.05 | 0.28 | 0.06 | 0.01 | 0.06 | 0.05 |
| | 8 T | 0.11 | 0.11 | 0.10 | 0.09 | 0.10 | 0.51 | 0.10 | 0.01 | 0.11 | 0.09 |
| | 8 U | 0.06 | 0.07 | 0.06 | 0.06 | 0.06 | 0.31 | 0.06 | 0.01 | 0.07 | 0.06 |
| | 9 T | 0.10 | 0.11 | 0.10 | 0.10 | 0.11 | 0.52 | 0.10 | 0.01 | 0.11 | 0.10 |
| | 9 U | 0.07 | 0.07 | 0.07 | 0.06 | 0.06 | 0.33 | 0.07 | 0.01 | 0.07 | 0.06 |
| | 10 T | 0.10 | 0.10 | 0.09 | 0.09 | 0.10 | 0.48 | 0.10 | 0.01 | 0.10 | 0.09 |
| | 10 U | 0.07 | 0.06 | 0.06 | 0.06 | 0.08 | 0.31 | 0.06 | 0.01 | 0.07 | 0.06 |
| | 11 T | 0.11 | 0.09 | 0.11 | 0.10 | 0.11 | 0.52 | 0.10 | 0.01 | 0.11 | 0.09 |
| | 11 U | 0.07 | 0.07 | 0.06 | 0.07 | 0.06 | 0.33 | 0.07 | 0.01 | 0.07 | 0.08 |
| | 12 T | 0.11 | 0.11 | 0.10 | 0.10 | 0.09 | 0.51 | 0.10 | 0.01 | 0.11 | 0.09 |
| | 12 U | 0.06 | 0.05 | 0.06 | 0.05 | 0.06 | 0.28 | 0.06 | 0.01 | 0.06 | 0.05 |
| C | 13 T | 0.09 | 0.09 | 0.08 | 0.10 | 0.09 | 0.45 | 0.09 | 0.01 | 0.10 | 0.08 |
| | 13 U | 0.06 | 0.07 | 0.06 | 0.05 | 0.06 | 0.30 | 0.06 | 0.01 | 0.07 | 0.05 |
| | 14 T | 0.10 | 0.10 | 0.09 | 0.09 | 0.09 | 0.47 | 0.09 | 0.01 | 0.10 | 0.09 |
| | 14 U | 0.06 | 0.06 | 0.07 | 0.08 | 0.06 | 0.31 | 0.06 | 0.01 | 0.07 | 0.06 |
| | 15 T | 0.10 | 0.08 | 0.08 | 0.10 | 0.09 | 0.45 | 0.09 | 0.01 | 0.10 | 0.08 |
| | 15 U | 0.07 | 0.06 | 0.06 | 0.07 | 0.05 | 0.31 | 0.06 | 0.01 | 0.07 | 0.05 |
| | 16 T | 0.09 | 0.08 | 0.09 | 0.09 | 0.08 | 0.43 | 0.09 | 0.01 | 0.09 | 0.08 |
| | 16 U | 0.05 | 0.05 | 0.06 | 0.06 | 0.06 | 0.28 | 0.06 | 0.01 | 0.06 | 0.05 |
| | 17 T | 0.09 | 0.09 | 0.08 | 0.09 | 0.09 | 0.44 | 0.09 | 0.01 | 0.09 | 0.08 |
| | 17 U | 0.06 | 0.06 | 0.07 | 0.05 | 0.06 | 0.30 | 0.05 | 0.01 | 0.07 | 0.05 |
| | 18 T | 0.09 | 0.09 | 0.07 | 0.08 | 0.10 | 0.43 | 0.09 | 0.02 | 0.10 | 0.07 |
| | 18 U | 0.07 | 0.06 | 0.06 | 0.07 | 0.07 | 0.33 | 0.07 | 0.01 | 0.07 | 0.06 |
| D | 19 T | 0.06 | 0.07 | 0.06 | 0.05 | 0.06 | 0.30 | 0.06 | 0.01 | 0.07 | 0.05 |
| | 19 U | 0.06 | 0.05 | 0.05 | 0.06 | 0.06 | 0.28 | 0.06 | 0.01 | 0.06 | 0.05 |
| | 20 T | 0.05 | 0.07 | 0.07 | 0.06 | 0.06 | 0.31 | 0.06 | 0.01 | 0.07 | 0.05 |
| | 20 U | 0.06 | 0.05 | 0.04 | 0.06 | 0.05 | 0.26 | 0.05 | 0.01 | 0.06 | 0.04 |
| | 21 T | 0.07 | 0.06 | 0.06 | 0.07 | 0.07 | 0.33 | 0.07 | 0.01 | 0.07 | 0.06 |
| | 21 U | 0.06 | 0.06 | 0.06 | 0.06 | 0.06 | 0.30 | 0.06 | 0.00 | 0.08 | 0.06 |
| | 22 T | 0.06 | 0.06 | 0.07 | 0.06 | 0.06 | 0.31 | 0.06 | 0.01 | 0.07 | 0.06 |
| | 22 U | 0.06 | 0.05 | 0.05 | 0.06 | 0.06 | 0.28 | 0.06 | 0.01 | 0.06 | 0.05 |
| | 23 T | 0.05 | 0.07 | 0.06 | 0.06 | 0.07 | 0.31 | 0.06 | 0.01 | 0.07 | 0.05 |
| | 23 U | 0.06 | 0.06 | 0.05 | 0.06 | 0.06 | 0.29 | 0.06 | 0.01 | 0.06 | 0.05 |
| | 24 T | 0.06 | 0.06 | 0.07 | 0.07 | 0.06 | 0.32 | 0.06 | 0.01 | 0.07 | 0.06 |
| | 24 U | 0.07 | 0.06 | 0.06 | 0.06 | 0.07 | 0.32 | 0.06 | 0.01 | 0.07 | 0.06 |

### C. Ultrasound Studies

In the treated skin samples obtained immediately after mask material removal, disorganization was observed in the distribution of the collagen fibers in the surface dermal connective tissue. This disorganization was due to perifibrillar deposits of amorphous materials, probably water, imbibing the fundamental substance of connective tissue (FIGS. 10 and 11). In samples obtained later, no substantial changes were observed in the collagen fascicles disposition nor in connective tissue matrix.

D1. Immunohistoehemical Study of Type III Collagen

Both the treated and untreated skin samples showed uniform dermal Type III collagen distribution in the dermal papillae, with no significant differences in its amounts when evaluated per surface unit.

Nonetheless, differences were found in total amounts of Type III collagen. This difference was due to a modification in the shape of the dermal papilae in treated skin, papilae were flat and had a large base, whereas in untreated skin samples theft height was twofold greater than the width of their base. These changes were particularly sensed only in the samples obtained immediately after test mask material removal and overtime tended to lower until becoming practically indistinguishable (FIGS. 12 and 13).

D2. Immunohistochemical Study:

Heat Shock Protein (HSP) Expression

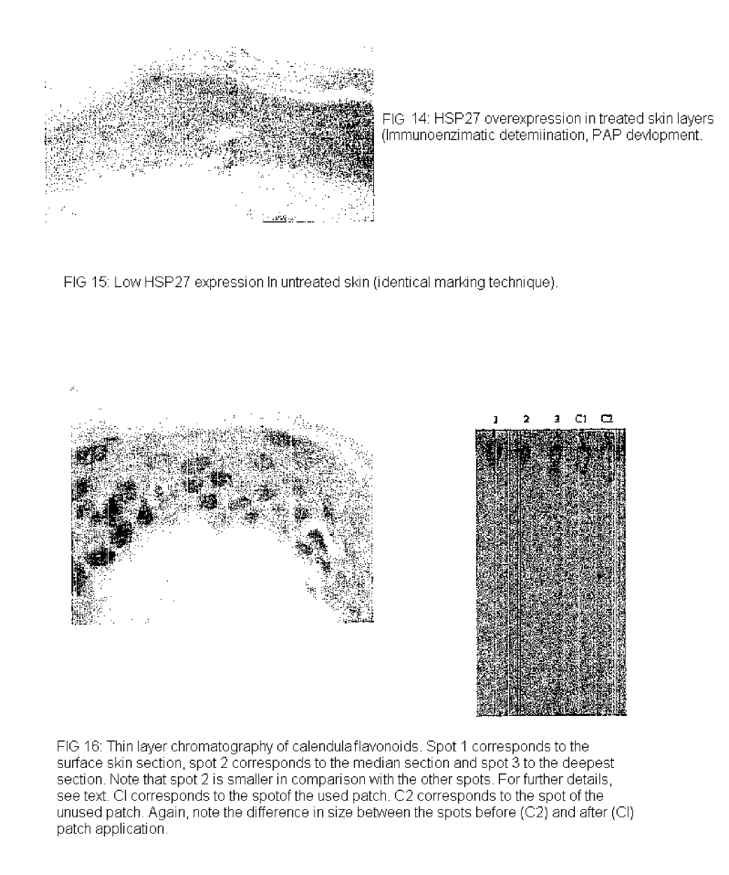

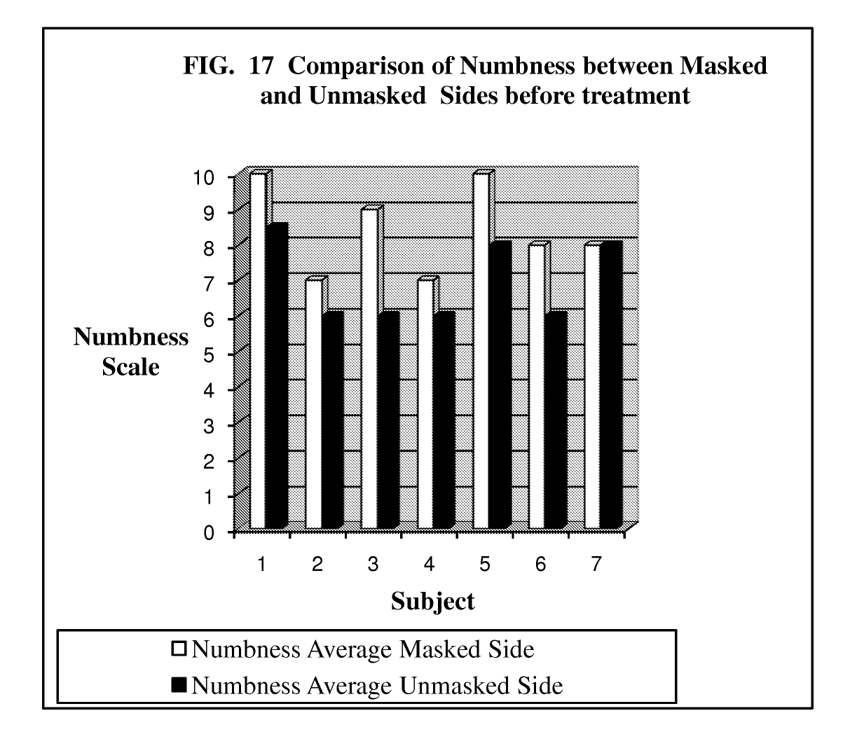

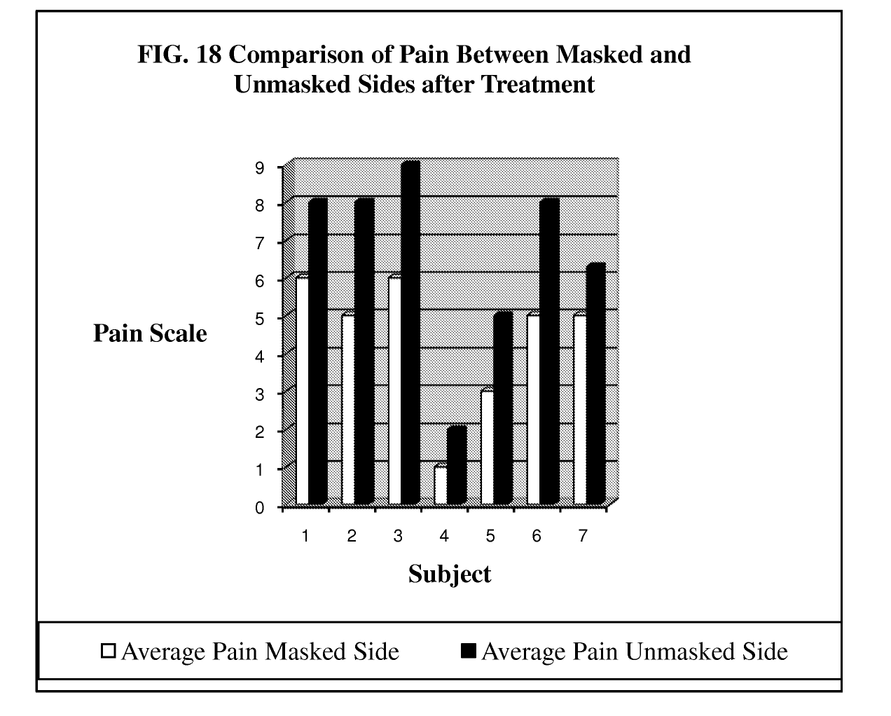

Treated skin samples obtained immediately after the removal of the test mask material showed a marked HSP27 over expression in the malpighian stratum. The expression of HSP70, HSP84 and HPS was similar between treated and untreated skin samples, and in both cases there were few areas in the malpighian layers. These differences in HSP27 expression remained in the sample one hour after test material removal, whereas in samples obtained later HSP27 over expression was no longer detected (FIGS. 14 and 15). Results are summarized in Table V.

TABLE V
Absorption Studies
Heat shock protein (HSP) expression in epidermis treated with the
mask material at different times after being removed vs. untreated
epidermis (GROUP A: samples obtained immediately after material
removal; GROUP B: samples obtained 1 hour after material removal;
GROUP C: samples obtained 3 hours after material removal;
GROUP D: samples obtained 6 hours after material removal)

| | | HSP27 | | HSP70 | | HSP84 | | HSP104 | |
|---|---|---|---|---|---|---|---|---|---|
| | VOL | T | U | T | U | T | U | T | U |
| A | 1 | +++ | +/− | + | + | + | +/− | +/− | + |
| | 2 | ++ | | +/− | +/− | +/− | | +/− | |
| | 3 | +++ | +/− | +/− | +/− | +/− | +/− | +/− | +/− |
| | 4 | +++ | + | | +/− | +/− | | | |
| | 5 | +++ | +/− | +/− | +/− | +/− | +/− | | |
| | 6 | +++ | + | + | + | +/− | + | 1 +/− | +/− |
| B | 7 | ++ | +/− | +/− | +/− | +/− | + | | |
| | 8 | ++ | +/− | | +/− | + | +/− | +/− | +/− |
| | 9 | +++ | + | + | +/− | +/− | +/− | | +/− |
| | 10 | ++ | | +/− | +/− | +/− | | +/− | |
| | 11 | ++ | + | +/− | +/− | +/− | +/− | +/− | +/− |
| | 12 | ++ | +/− | + | + | +/− | +/− | | |
| C | 13 | ++ | +/− | + | +/− | +/− | + | +/− | +/− |
| | 14 | + | +/− | +/− | +/− | +/− | +/− | +/− | +/− |
| | 15 | + | | +/− | +/− | +/− | +/− | +/− | +/− |
| | 16 | ++ | + | +/− | | +/− | +/− | +/− | +/− |
| | 17 | ++ | +/− | | | +/− | +/− | +/− | |
| | 18 | + | | +/− | +/− | +/− | +/− | | +/− |
| D | 19 | +/− | | +/− | | +/− | + | | +/− |
| | 20 | + | +/− | +/− | | | +/− | +/− | +/− |
| | 21 | +/− | +/− | | +/− | + | +/− | +/− | |
| | 22 | | +/− | | +/− | +/− | | | |
| | 23 | +/− | | +/− | +/− | +/− | +/− | +/− | +/− |
| | 24 | | | +/− | +/− | +/− | +/− | | |

(−): No expression
+/−: Minimal expression
+: Low expression
++: Normal expression
+++: Marked expression
T: Treated skin
U: Untreated skin
VOL: Volunteers

Thin layer chromatography revealed a greenish spot that fluoresced when exposed to UV light at wavelengths 254 to 366 nanometers, with a response factor of (RI) of 0.2±0.04 in all elutes from sections made at different skin tissue depths. Its intensity decreased in sample n°2, from medium skin section (sample identified as n°1 corresponds to surface skin section, of 0 to 100 m in thickness, and sample n°3 corresponds to deep section, from 200 to 300 m thickness). This chromatography profile could only be observed in samples obtained immediately after test material removal. No spot was detected in sections made at depths in subsequent samples (FIG. 16).

An exemplary composition mixture of the present invention is as follows:

| | Mixed | Percent | Solids | Dry | Wet | Quantity | Dry |
|---|---|---|---|---|---|---|---|
| | Quantity | Solids | Content | Weight | Weight | per Batch | Parts |
| PVOH | 12.2429 | 99.7 | 12.206 | 64.405 | 12.24 | 146.91 | 100 |
| NEO-BEE | 2.1663 | 99.9 | 2.163 | 11.414 | 2.17 | 25.984 | 17.7 |
| M-20 PG-865 | | | | | | | |
| PHOSPHOLIPID | 1.7804 | 49 | 0.872 | 4.603 | 1.78 | 21.365 | 7.1 |
| EFA | | | | | | | |
| LEXQUAT C | 2.2507 | 36 | 0.810 | 4.275 | 2.25 | 27.008 | 6.6 |
| CITRIC ACID | 2.0192 | 25 | 0.505 | 2.664 | 2.02 | 24.231 | 4.1 |
| (25%) | | | | | | | |
| GLYCOLIC ACID | 0.5099 | 99 | 0.505 | 2.663 | 0.51 | 6.118 | 4.1 |
| (99%) | | | | | | | |
| LACTIC Acid | 0.5608 | 90 | 0.505 | 2-663 | 0.56 | 6.729 | 4.1 |
| (90%) | | | | | | | |
| NaOH Premix | 3.7333 | 10 | 0.373 | 1.970 | 3.73 | 44.799 | 3.1 |
| HONEY | 0.3694 | 96 | 0.355 | 1.871 | 0.37 | 4.433 | 2.9 |
| SODIUM PCA | 0.3920 | 50 | 0.196 | 1.034 | 0.39 | 4.704 | 1.6 |
| AJIDEW N-50 | | | | | | | |
| VITAMIN E | 0.1950 | 96 | 0.187 | 0.988 | 0.195 | 2.340 | 1.5 |
| ACETATE | | | | | | | |
| VITAMIN A | 0.1027 | 96 | 0.099 | 0.520 | 0.10 | 1.232 | 0.8 |
| PALMITATE USP | | | | | | | |
| Sodium Ascorbyl | 0.1027 | 94 | 0.097 | 0.509 | 0.10 | 1.232 | 0.8 |
| Phosphate | | | | | | | |
| Biotanical | 0.3989 | 20 | 0.080 | 0.421 | 0.40 | 4.787 | 0.7 |
| Extracts | | | | | | | |
| PURIFIED | 73.1769 | 0 | 0.000 | 0.000 | 73.18 | 878.123 | 0.0 |
| WATER | | | | | | | |
| Totals g | 100.0000 | | 18.952 | 100.000 | 100.00 | 1200 | 155.3 |

Polyvinyl Alcohol Film will dry to 5-11.4% moisture (=water) propylene glycol dicaprylate dicaprate

Discussion

In view of these results, it can be said that this type of skin product delivery system has the potential of delivering a multitude of ingredients to improve cutaneous texture. One of the major skin changes observed includes stratum corneum broadening, due to rehydration and water retention, considering the moisture absorption increase of treated skin. This corneum thickening expansion is not due to an increase in the number of corneum layers, but to stratum corneum divulsion, which is likely due to water accumulation between the layers. Furthermore, it must be taken into account that stratum corneum surface layers are exfoliated when the test materials are removed, as shown by the study on the concentration of corneum layers that remain adhered to the test material. This exfoliative action was evident in previous clinical assays, explaining the smooth aspect of the cutaneous surface.

On the other hand, modification of the cutaneous texture could also be due to changes in the shape of the dermal papillae, the broadening of their base as well as the lowering of the height. This modification could be due to water accumulation between collagen fiber fascicles, as inferred from the ultrastructural and immunochemical studies of Type III collagen distribution result.

Another important change was the vascular diameters in the treated skin in the surface dermal plexus. Vascular diameters in the treated skin increased from 30 to 50% as compared to untreated skin. This change leads to a slower blood flow with decrease in intravascular hydrostatic pressure, not favoring the absorption of extracellular liquid retained in the area surrounding the vessels, which correlates with these observations. On the other hand this vasodilation leads to local increases in temperature, which favors an increase in cellular metabolic processes. The skin has a temperature-dependent metabolism differ from the rest of the body, since change in ambient temperature influences skin metabolism. The same does not hold true of internal tissues, which are thermal isolated by adipose tissues in the hypodermis and usually function at temperature close to 37° C. These increases of vascular diameter and subsequent rises in local temperature were measured thermographical.

In addition, there is a physical process to be considered: the mask material functions as an isolated item, preventing the evaporation of water which would cool down the skin surface, creating an interface which preserves the moisture and generating a homogeneous microclimate not subject to ambient temperatures. The absorbed water retained in the corneum layer also may function as an isolating barrier, preventing or reducing heat diffusion to the exterior.

A possible consequence of the later, the heat shock protein (HSP) type 27 is over expressed in the treated epidermis, while different molecular weights of 70, 84 and 104 show no significant expression. Heat shock proteins are usually activated by a series of stimuli, including heat heavy metals, and methanol. HPS84 and HPS 27 expression occurs in both stress and non stress conditions, since these proteins are constitutively expressed in normal cells, particularly in association with other structural proteins such as tublin and actin because both proteins like keratin and/or its synthesis system, it may be considered that the observed over expression could be due to unmasked HSP27 antigenic determinates associated with the tubinlin-keratin protein complex.

There is also the possibility that the over expression is caused by the absorbed constituents of the mask material. This over expression indicates the existence of a change in keratinocyte homeostasis. These studies also indicate the skin permeability is unaltered since calendula flavonoids were detected in deep as well as surface sections of skin. These studies tend to prove that the skin remains permeable with the flavonoids observed in the surface layers and are probably retained in the keratin, somehow, while the excess flavonoids observed in the deeper layers could be due to a passage of excess flavonoids after saturation of epidermal retention capacity is reached. The absence of flavonoids in median sections of the skin is interpreted as a mere question of timing in absorption kinetics. If mask materials were applied for 20 minutes or 3 hours instead of 45 minutes, the chromatogram profile may have been different.

In view of these results achieved at different times after mask test materials application, it is evident that the intensity of changes described decrease over time, though clinical trials have shown that the effects last several hours.

The results achieved produced clinically evident and experimentally measurable effects through a mechanism of an action far from complex. Once the mask material was activated and in contact with wet skin, the occlusive layer appears to interfere with the process of air exchange, increasing the local temperature, blocking the evaporation and creating a new microenvironment over the occluded skin which leads to a capillary dilation creating a pushing/pulling effect that literally pushes water to the inside surface strata, adding to water accumulation due to the subsequent vascular phenomena.

Aside from the physical phenomena related to the structure of the mask material, the constituents in the formulation help product the observed effects. The presence of sodium PCA in the formula increases the process of water capture, contributing to the overall water retention in the tissues. The presence of phospholipids and flavonoids complement the mechanism of action of the mask material, by providing moisture and critical proteins to the cell structure which promotes faster absorption of the other active ingredients.

Finally, the exfoliative effect of these patches is worth noting. Even during the clinical tests, a distinct “peeling” of the skin surface could be noticed, as if it had suffered a slight abrasion. Confirmation of the exfoliative effect was obtained through study of the cells adhered to the patch after use. Exfoliation was higher as expected, on those skin areas, which due to their biological functions, have a higher metabolic rate, such as the facial areas. Even though facial skin is popularly regarded as the most sensitive skin, its evolutive function makes it in fact the most resilient. It should be remembered that it is subject to all the climatic and environmental changes that in some it has little or no protective hair. Thus, the facial skin has a higher metabolic rate than other types of skin. This is especially apparent when considering sebaceous hypersecretion localized of the face and neckline triangle, which provides a special lubrication and thermal protection to these skin areas.

The increased exfoliative effect of instant invention on facial areas allows removal of the uppermost, surface corneum layers of the epidermis, sweeping with them the lipid layer were all the metabolic detritus of the skin are deposited, together with saprophytic bacteria and environmental contaminants. All these elements have an oxidizing effect on the lipids, which gives the skin a dull and opaque look. Removal of this oxidized lipid layer and of the surface strata from the corneum layers could be the reason behind the peculiar luminescence of instant invention treated skin observed in the clinical tests. This effect appears to be related to a deep cleansing of the epidermis.

The thinning-out of the treated sebhorreic keratoses is also related to this exfoliative mechanism. These exfoliative effects are more noticeable in sebhorreic skins. The local temperature increase observed could have a favorable effect on metabolic reactivation of treated skin. This exfoliative mechanism is possibly no more than a physical phenomenon of adherence of the surface layers of the skin to the patch/mask, given that this effect is generalized and uniform throughout the treated area.

For this reason, the possibility that one of the botanical components of the instant invention could contain natural substances with exfoliative properties should not be disregarded. Discussion of the biological effects of the many extracts that constitute the patch/mask exceeds the objectives of this study, even though it is known that many of them have an exfoliative effect. Further information regarding mechanism of action of the botanical extracts can be found in specialized literature.

A separate and distinct benefit has been discovered through the use of the instant invention. In this regard, a mask 10 which includes an exemplary composition as described above has significant benefit in alleviating pain when administering treatment to the skin, such as facial skin. In this regard, the mask 10 and composition aids in driving anesthetic into the skin and thus when removed permits laser treatments or other composition treatments to be performed which without the use of the mask 10 would be significantly more painful. This aids the speed in which the process can be performed on a patient.

Results of an empirical study yield the following results. The following Tables shows various results from application of the mask of the invention. Table VI is one study with use of Lidocaine and the effectiveness with respect to reduced pain level where pain/numbness was on a 1-10 scale with 1 being least pain and 10 highest pain level wherein a significant pain reduction and enhanced numbness was noticed by patients using the mask of the instant invention.

TABLE VI

| | | | | | Time |
|---|---|---|---|---|---|
| | | | | | Lidocaine |
| | Last Name | | | | Applied |
| Patient | Init. | First Name Init. | Procedure | Mask | (Left Side) |
| 1 | L | P | Fraxel | Left | 1:40 PM |
| 2 | J | D | Fraxel | Right | 2:40 PM |
| 3 | A | N | Fraxel | Left | N/A |
| 4 | K | L | Fraxel | Left | N/A |
| 5 | S | J | CO2 | Left | N/A |
| 6 | A | B | CO2 | Right | 12:30 PM |
| 7 | S | L | CO2 | Left | N/A |
| 8 | W | V | CO2 | Right | 12:05 PM |
| 9 | M | D | CO2 | Left | N/A |
| 10 | P | L | CO2 | Left | 1:15 PM |
| 11 | S | G | CO2 | Left | N/A |
| 12 | O | J | CO2 | Right | 2:00 PM |

| | Time | Time | Time | | |
|---|---|---|---|---|---|
| | Lidocaine | Lidocaine + Mask | Lidocaine + Mask | Numbness- | Numbness |
| | Applied | Applied (Left | Applied (Right | Mouth | Cheeks- |
| Patient | (Right Side) | Side) | Side) | (Left Side) | (Left Side) |
| 1 | N/A | 2:00 PM | — | 2 | 4 |
| 2 | N/A | — | 2:45 PM | 4 | 4 |
| 3 | 2:30 PM | 2:35 PM | — | 5 | 5 |
| 4 | 3:50 PM | 3:40 PM | — | 7 | 7 |
| 5 | 2:10 PM | 2:20 PM | — | 8 | 8 |
| 6 | N/A | — | 12:45 PM | 8 | 8 |
| 7 | 2:40 PM | 2:45 PM | — | 6 | 6 |
| 8 | N/A | — | 12:07 PM | 9 | 9 |
| 9 | 11:06 AM | 11:00 AM | — | | |
| 10 | N/A | 1:20 PM | — | 7 | 7 |
| 11 | 2:05 PM | 2:07 PM | — | 7 | 7 |
| 12 | N/A | — | 2:06 PM | 5 | 3 |

| | Numbness- | Numbness- | Numbness- | Numbness | Numbness- |
|---|---|---|---|---|---|
| | Forehead | Chin (Left | Mouth (Right | Cheeks- (Right | Forehead |
| Patient | (Left Side) | Side) | Side) | Side) | (Right Side) |
| 1 | 2 | 2 | 5 | 7 | 4 |
| 2 | 4 | 4 | 6 | 6 | 6 |
| 3 | 4 | 5 | 3 | 3 | 3 |
| 4 | 8 | 7 | 6 | 5 | 6 |
| 5 | 8 | 8 | 7 | 7 | 7 |
| 6 | 8 | 8 | 7 | 7 | 7 |
| 7 | 6 | 6 | 4 | 4 | 4 |
| 8 | 9 | 9 | 9 | 9 | 9 |
| 9 | | | | | |
| 10 | 7 | 7 | 6 | 6 | 6 |
| 11 | 8 | 6 | 8 | 7 | 8 |
| 12 | 2 | 7 | 4 | 4 | 3 |

| | Numbness- | Difference | | Pain After | Pain After |
|---|---|---|---|---|---|
| | Chin (Right | between R | Time of | proc. Mouth | proc. Cheeks |
| Patient | Side) | and L side? | Procedure | (Left Side) | (Left Side) |
| 1 | 3 | Yes | N/A | 5 | 5 |
| 2 | 6 | Yes | N/A | N/A | N/A |
| 3 | 4 | No | N/A | 10 | 7 |
| 4 | 5 | Yes | N/A | 9 | 9 |
| 5 | 7 | No | N/A | N/A | N/A |
| 6 | 7 | No | N/A | 10 | 10 |
| 7 | 4 | Yes | N/A | 8 | 8 |
| 8 | 9 | No | N/A | 6 | 6 |
| 9 | | | | | |
| 10 | 6 | Yes | N/A | 4 | 4 |
| 11 | 7 | No | N/A | 10 | 10 |
| 12 | 8 | Yes | N/A | 4 | 5 |

| | Pain After | Pain After | Pain After | Pain After | Pain After |
|---|---|---|---|---|---|
| | proc. Forehead | proc. Chin | proc. Mouth | proc. Cheeks | proc. Forehead |
| Patient | (Left Side) | (Left Side) | (Right Side) | (Right Side) | (Right Side) |
| 1 | 6 | 6 | 4 | 4 | 4 |
| 2 | N/A | N/A | N/A | N/A | N/A |
| 3 | 10 | 6 | 10 | 7 | 10 |
| 4 | 10 | 9 | 8 | 8 | 8 |
| 5 | N/A | N/A | N/A | N/A | N/A |
| 6 | 10 | 10 | 10 | 10 | 10 |
| 7 | 8 | 8 | 9 | 9 | 9 |
| 8 | 6 | 6 | 6 | 6 | 6 |
| 9 | | | | | |
| 10 | 4 | 6 | 4 | 9 | 4 |
| 11 | 10 | 10 | 10 | 10 | 10 |
| 12 | 6 | 5 | 4 | 7 | 9 |

| | Pain After | Application | Application | Feeling | Feeling durind |
|---|---|---|---|---|---|
| | proc. Chin | Process | Process | durind appl. | appl. |
| Patient | (Right Side) | Easy | complicated | Comfortable | Uncomfortable |
| 1 | 4 | yes | no | yes | no |
| 2 | N/A | N/A | N/A | N/A | N/A |
| 3 | 6 | yes | no | yes | no |
| 4 | 8 | yes | no | yes | no |
| 5 | N/A | N/A | N/A | N/A | N/A |
| 6 | 10 | N/A | N/A | N/A | N/A |
| 7 | 9 | N/A | N/A | N/A | N/A |
| 8 | 6 | N/A | N/A | N/A | N/A |
| 9 | | N/A | N/A | N/A | N/A |
| 10 | 6 | N/A | N/A | N/A | N/A |
| 11 | 10 | N/A | N/A | N/A | N/A |
| 12 | 8 | N/A | N/A | N/A | N/A |

| | | Taking the | | | |
|---|---|---|---|---|---|
| | Taking the | Mask | Dryness | Dryness | |
| Patient | mask off Easy | Difficult | Right side | Left side | Burning Right |
| 1 | yes | no | no | no | no |
| 2 | N/A | N/A | N/A | N/A | N/A |
| 3 | yes | no | no | no | no |
| 4 | yes | no | no | no | yes |
| 5 | N/A | N/A | N/A | N/A | N/A |
| 6 | N/A | N/A | N/A | N/A | N/A |
| 7 | N/A | N/A | N/A | N/A | N/A |
| 8 | N/A | N/A | N/A | N/A | N/A |
| 9 | N/A | N/A | N/A | N/A | N/A |
| 10 | N/A | N/A | N/A | N/A | N/A |
| 11 | N/A | N/A | N/A | N/A | N/A |
| 12 | N/A | N/A | N/A | N/A | N/A |

| | | Redness | | Itching | |
|---|---|---|---|---|---|
| Patient | Burning Left | Right | Redness Left | Right | Itching Left |
| 1 | no | yes | yes | no | no |
| 2 | N/A | N/A | N/A | N/A | N/A |
| 3 | no | no | no | no | no |
| 4 | yes | yes | yes | no | no |
| 5 | N/A | N/A | N/A | N/A | N/A |
| 6 | N/A | N/A | N/A | N/A | N/A |
| 7 | N/A | N/A | N/A | N/A | N/A |
| 8 | N/A | N/A | N/A | N/A | N/A |
| 9 | N/A | N/A | N/A | N/A | N/A |
| 10 | N/A | N/A | N/A | N/A | N/A |
| 11 | N/A | N/A | N/A | N/A | N/A |
| 12 | N/A | N/A | N/A | N/A | N/A |

| | Stinging | |
|---|---|---|
| Patient | Right | Stinging Left |
| 1 | yes | yes |
| 2 | N/A | N/A |
| 3 | no | no |
| 4 | yes | yes |
| 5 | N/A | N/A |
| 6 | N/A | N/A |
| 7 | N/A | N/A |
| 8 | N/A | N/A |
| 9 | N/A | N/A |
| 10 | N/A | N/A |
| 11 | N/A | N/A |
| 12 | N/A | N/A |

TABLE VII
Results with Lidocaine

| | | | | | | Pain | |
|---|---|---|---|---|---|---|---|
| | | | | | | After | |
| | | | | | | proc. | |
| | Difference | Side | Pain After | Pain After | Pain After | Chin | Average |
| | between R | with | proc. Mouth | proc. Cheeks | proc. Forehead | (Left | Left |
| Patient | and L side? | Mask | (Left Side) | (Left Side) | (Left Side) | Side) | Side |
| 1 | Yes | Left | 5 | 5 | 6 | 6 | 5.50 |
| 2 | Yes | Right | | | | | |
| 3 | No | Left | 10 | 7 | 10 | 6 | 8.25 |
| 4 | Yes | Left | 9 | 9 | 10 | 9 | 9.25 |
| 5 | No | Left | | | | | |
| 6 | No | Right | 10 | 10 | 10 | 10 | 10.00 |
| 7 | Yes | Left | 8 | 8 | 8 | 8 | 8.00 |
| 8 | No | Right | 6 | 6 | 6 | 6 | 6.00 |
| 9 | | Left | | | | | |
| 10 | Yes | Left | 4 | 4 | 4 | 6 | 4.50 |
| 11 | No | Left | 10 | 10 | 10 | 10 | 10.00 |
| 12 | Yes | Right | 4 | 5 | 6 | 5 | 5.00 |

| | Pain After | | | | |
|---|---|---|---|---|---|
| | proc. Mouth | Pain After | Pain After | Pain After | |
| | (Right | proc. Cheeks | proc. Forehead | proc. Chin | Average Right |
| Patient | Side) | (Right Side) | (Right Side) | (Right Side) | Side |
| 1 | 4 | 4 | 4 | 4 | 4.00 |
| 2 | | | | | |
| 3 | 10 | 7 | 10 | 6 | 8.25 |
| 4 | 8 | 8 | 8 | 8 | 9.25 |
| 5 | | | | | |
| 6 | 10 | 10 | 10 | 10 | 10.00 |
| 7 | 9 | 9 | 9 | 9 | 9.00 |
| 8 | 6 | 6 | 6 | 6 | 6.00 |
| 9 | | | | | |
| 10 | 4 | 9 | 4 | 6 | 4.50 |
| 11 | 10 | 10 | 10 | 10 | 10.00 |
| 12 | 4 | 7 | 9 | 8 | 5.00 |

Pain Rate

| | | | | | Numbness |
|---|---|---|---|---|---|
| | | | Numbness | | After |
| | Difference | | Before | Numbness | Proc. |
| | between R | | Proc. | Before proc. | Forehead |
| | and L | Side with | Mouth (Left | Cheeks (Left | (Left |
| Patient | side? | Mask | Side) | Side) | Side) |
| 1 | Yes | Left | | | |
| 2 | Yes | Left | | | |
| 3 | Yes | Left | | | |
| 4 | Yes | Left | | | |

| | | | | | Numbness | |
|---|---|---|---|---|---|---|
| | Numbness | | Numbness | | After | Numbness |
| | After | | Before | Numbness | Proc. | After |
| | proc. Chin | | Proc. | Before proc. | Forehead | proc. Chin |
| | (Left | Average | Mouth | Cheeks (Right | (Left | (Left |
| Patient | Side) | Left Side | (Right Side) | Side) | Side) | Side) |
| 1 | | | | | | |
| 2 | | | | | | |
| 3 | | | | | | |
| 4 | | | | | | |

| | | | | Pain After | Pain After |
|---|---|---|---|---|---|
| | | Pain After | Pain After | proc. Fore- | proc. Chin |
| | Average | proc. Mouth | proc. Cheeks | head (Left | (Left |
| Patient | Left Side | (Left Side) | (Left Side) | Side) | Side) |
| 1 | | 6 | 6 | 6 | 6 |
| 2 | | 8 | 8 | 8 | 8 |
| 3 | | 6 | 6 | 6 | 6 |
| 4 | | 1 | 0 | 1 | 1 |

| | | Pain After | | | Pain After | |
|---|---|---|---|---|---|---|
| | | proc. Mouth | Pain After | Pain After | proc. Chin | Average |
| | Average | (Right | proc. Cheeks | proc. Forehead | (Right | Right |
| Patient | Left Side | Side) | (Right Side) | (Right Side) | Side) | Side |
| 1 | 6.00 | 8 | 8 | 8 | 8 | 8.00 |
| 2 | 8.00 | 5 | 5 | 5 | 5 | 5.00 |
| 3 | 6.00 | 9 | 9 | 9 | 9 | 9.00 |
| 4 | 0.75 | 2 | 2 | 2 | 2 | 2.00 |

TABLE VIII
Pain Data

| | | Pain After | Pain After | Pain After | | Pain After | Pain After | Pain After | Pain After | |
|---|---|---|---|---|---|---|---|---|---|---|
| | Pain After proc. | proc. | proc. | proc. | Average | proc. | proc. | proc. | proc. | |
| | Mouth | Cheeks | Forehead | Chin | Pain | Mouth | Cheeks | Forehead | Chin | Average Pain |
| | (Masked | (Masked | (Masked | (Masked | (Masked | (Unmasked | (Unmasked | (Unmasked | (Unmasked | (Unmasked |
| Patient | Side) | Side) | Side) | Side) | Side) | Side) | Side) | Side) | Side) | Side) |
| 1 | 6 | 6 | 6 | 6 | 6 | 8 | 8 | 8 | 8 | 8 |
| 2 | 5 | 5 | 5 | 5 | 5 | 8 | 8 | 8 | 8 | 8 |
| 3 | 6 | 6 | 6 | 6 | 6 | 9 | 9 | 9 | 9 | 9 |
| 4 | 1 | 0 | 1 | 1 | 0.8 | 2 | 2 | 2 | 2 | 2 |
| 5 | 3 | 3 | 3 | 3 | 3 | 5 | 5 | 5 | 5 | 5 |
| 6 | 5 | 5 | 5 | 5 | 5 | 8 | 8 | 8 | 8 | 8 |
| 7 | 5 | 5 | 5 | 5 | 5 | 6 | 6 | 7 | 6 | 6.3 |

Table IX shows comparative data for a group of patients using the mask of the invention.

TABLE IX

| Patient | Date | D.O.B. | Age | Race | Ethnicity |
|---|---|---|---|---|---|
| 1 | Jan. 29, 2009 | Nov. 8, 1947 | 61 | Hispanic | White |
| 2 | Jan. 29, 2009 | Jan. 2, 1981 | 28 | Not Hisp. | White |
| 3 | Jan. 29, 2009 | Oct. 1, 1954 | 54 | Hispanic | White |
| 4 | Jan. 29, 2009 | May 11, 1950 | 58 | Not Hisp. | White |
| 5 | Feb. 26, 2009 | Jul. 6, 1970 | 38 | Not Hisp. | White |
| 6 | Feb. 26, 2009 | Dec. 9, 1942 | 66 | Not Hisp. | White |
| 7 | Feb. 26, 2009 | Jul. 11, 1936 | 73 | Not Hisp. | White |

| | | | | | Numbness- |
|---|---|---|---|---|---|
| | | | | | Mouth |
| Patient | Procedure | Mask Side | Mask Side | Other Side | (Masked Side) |
| 1 | CO2 | Left | 13:50 PM | 13:40 PM | 10 |
| 2 | CO2 | Left | 14:10 PM | 14:15 PM | 7 |
| 3 | CO2 | Left | 14:50 PM | 14:55 PM | 9 |
| 4 | CO2 | Left | 15:15 PM | 15:00 PM | 7 |
| 5 | CO2 | Right | 12:20 PM | 12:30 PM | 10 |
| 6 | CO2 | Right | 13:20 PM | 13:25 PM | 8 |
| 7 | CO2 | Right | 15:25 PM | 15:30 PM | 8 |

| | | | | Numbness- | Numbness |
|---|---|---|---|---|---|
| | Numbness | Numbness- | Numbness- | Mouth | Cheeks- |
| | Cheeks- | Forehead | Chin | (Unmasked | (Unmasked |
| Patient | (Masked Side) | (Masked Side) | (Masked Side) | Side) | Side) |
| 1 | 10 | 10 | 10 | 8 | 8 |
| 2 | 7 | 7 | 7 | 6 | 6 |
| 3 | 9 | 9 | 9 | 6 | 6 |
| 4 | 7 | 7 | 7 | 6 | 6 |
| 5 | 10 | 10 | 10 | 8 | 8 |
| 6 | 8 | 8 | 8 | 6 | 6 |
| 7 | 8 | 8 | 8 | 8 | 8 |

| | Numbness- | Numbness- | | Pain After | Pain After |
|---|---|---|---|---|---|
| | Forehead | Chin | Difference | proc. Mouth | proc. Cheeks |
| | (Unmasked | (Unmasked | between R | (Masked | (Masked |
| Patient | Side) | Side) | and L side? | Side) | Side) |
| 1 | 9 | 9 | Yes | 6 | 6 |
| 2 | 6 | 6 | Yes | 5 | 5 |
| 3 | 6 | 6 | Yes | 6 | 6 |
| 4 | 6 | 6 | Yes | 1 | 0 |
| 5 | 8 | 8 | Yes | 3 | 3 |
| 6 | 6 | 6 | Yes | 5 | 5 |
| 7 | 8 | 8 | No | 5 | 5 |

| | Pain After | | | | |
|---|---|---|---|---|---|
| | proc. | Pain After | Pain After | Pain After | Pain After |
| | Forehead | proc. Chin | proc. Mouth | proc. Cheeks | proc. Forehead |
| | (Masked | (Masked | (Unmasked | (Unmasked | (Unmasked |
| Patient | Side) | Side) | Side) | Side) | Side) |
| 1 | 6 | 6 | 8 | 8 | 8 |
| 2 | 5 | 5 | 8 | 8 | 8 |
| 3 | 6 | 6 | 9 | 9 | 9 |
| 4 | 1 | 1 | 2 | 2 | 2 |
| 5 | 3 | 3 | 5 | 5 | 5 |
| 6 | 5 | 5 | 8 | 8 | 8 |
| 7 | 5 | 5 | 6 | 6 | 7 |

| | Pain After | | | | |
|---|---|---|---|---|---|
| | proc. Chin | Application | Feeling | Taking the | |
| | (Unmasked | Process | during appl. | mask off | Adverse |
| Patient | Side) | Easy | Comfortable | Easy | events |
| 1 | 8 | Yes | yes | yes | No |
| 2 | 8 | Yes | yes | yes | No |
| 3 | 9 | Yes | yes | yes | No |
| 4 | 2 | Yes | yes | yes | No |
| 5 | 5 | Yes | yes | yes | No |
| 6 | 8 | Yes | yes | yes | No |
| 7 | 6 | Yes | yes | yes | No |

Table X shows comparative data for a group of patients using the mask of the invention.

TABLE X

| Patient | Date | Last init. | First init. | D.O.B. | Age |
|---|---|---|---|---|---|
| 1 | Jan. 29, 2009 | C | M | Nov. 8, 1947 | 61 |
| 2 | Jan. 29, 2009 | H | L | Jan. 2, 1981 | 28 |
| 3 | Jan. 29, 2009 | S | L | Oct. 1, 1954 | 54 |
| 4 | Jan. 29, 2009 | W | M | May 11, 1950 | 58 |
| 5 | Feb. 26, 2009 | R | K | Jul. 6, 1970 | 38 |
| 6 | Feb. 26, 2009 | S | M | Dec. 9, 1942 | 66 |
| 7 | Feb. 26, 2009 | K | J | Jul. 11, 1936 | 73 |

| Last init. | Race | Ethnicity | Procedure | Mask Side | Mask Side |
|---|---|---|---|---|---|
| C | Hispanic | White | CO2 | Left | 13:50:00 |
| | | | | | PM |
| H | Not | White | CO2 | Left | 14:10:00 |
| | Hispanic | | | PM | |
| S | Hispanic | White | CO2 | Left | 14:50:00 |
| | | | | | PM |
| W | Not | White | CO2 | Left | 15:15:00 |
| | Hispanic | | | PM | |
| R | Not | White | CO2 | Right | 12:20 PM |
| | Hispanic | | | | |
| S | Not | White | CO2 | Right | 13:20 PM |
| | Hispanic | | | | |
| K | Not | White | CO2 | Right | 15:25 PM |
| | Hispanic | | | | |

| | | Numbness- | Numbness | Numbness- | Numbness- |
|---|---|---|---|---|---|
| | | Mouth | Cheeks- | Forehead | Chin |
| | | (Masked | (Masked | (Masked | (Masked |
| Last init. | Other Side | Side) | Side) | Side) | Side) |
| C | 13:40:00 | 10 | 10 | 10 | 10 |
| | PM | | | | |
| H | 14:15:00 | 7 | 7 | 7 | 7 |
| | PM | | | | |
| S | 14:55:00 | 9 | 9 | 9 | 9 |
| | PM | | | | |
| W | 15:00 PM | 7 | 7 | 7 | 7 |
| R | 12:30 PM | 10 | 10 | 10 | 10 |
| S | 13:25 PM | 8 | 8 | 8 | 8 |
| K | 15:30 PM | 8 | 8 | 8 | 8 |
| | Numbness- | Numbness | Numbness- | Numbness- | |
| | Mouth | Cheeks- | Forehead | Chin | Difference |
| | (Unmasked | (Unmasked | (Unmasked | (Unmasked | between R |
| Last init. | Side) | Side) | Side) | Side) | and L side? |
| C | 8 | 8 | 9 | 9 | Yes |
| H | 6 | 6 | 6 | 6 | Yes |
| S | 6 | 6 | 6 | 6 | Yes |
| W | 6 | 6 | 6 | 6 | Yes |
| R | 8 | 8 | 8 | 8 | Yes |
| S | 6 | 6 | 6 | 6 | Yes |
| K | 8 | 8 | 8 | 8 | No |

| | Pain After | Pain After | Pain After | Pain After | Pain After |
|---|---|---|---|---|---|
| | proc. Mouth | proc. Cheeks | proc. Forehead | proc. Chin | proc. Mouth |
| | (Masked | (Masked | (Masked | (Masked | (Unmasked |
| Last init. | Side) | Side) | Side) | Side) | Side) |
| C | 6 | 6 | 6 | 6 | 8 |
| H | 5 | 5 | 5 | 5 | 8 |
| S | 6 | 6 | 6 | 6 | 9 |
| W | 1 | 0 | 1 | 1 | 2 |
| R | 3 | 3 | 3 | 3 | 5 |
| S | 5 | 5 | 5 | 5 | 8 |
| K | 5 | 5 | 5 | 5 | 6 |

| | Pain After | Pain After | Pain After | | |
|---|---|---|---|---|---|
| | proc. Cheeks | proc. Forehead | proc. Chin | Application | Feeling |
| | (Unmasked | (Unmasked | (Unmasked | Process | durind appl. |
| Last init. | Side) | Side) | Side) | Easy | Comfortable |
| C | 8 | 8 | 8 | yes | yes |
| H | 8 | 8 | 8 | yes | yes |
| S | 9 | 9 | 9 | yes | yes |
| W | 2 | 2 | 2 | yes | yes |
| R | 5 | 5 | 5 | yes | yes |
| S | 8 | 8 | 8 | yes | yes |
| K | 6 | 7 | 6 | yes | yes |

| | Taking the | | |
|---|---|---|---|
| Last Name | mask off | Adverse | |
| init. | Easy | events | Comments |
| C | yes | No | |
| H | yes | No | |
| S | yes | No | |
| W | yes | No | No pain at all in the cheek |
| | | | area (mask's side) |
| R | yes | No | |
| S | yes | No | |
| K | yes | No | |

Table XI shows results of the effective numbness of felt by patients using the mask of the invention with higher average numbness on 0-10 scale.

TABLE XI

| | Numbness | | | | |
|---|---|---|---|---|---|
| | Masked | | | | |
| | Side- | Numbness | Numbness- | Numbness- | |
| | Mouth | Cheeks- | Forehead | Chin | |
| | (Left | (Masked | (Masked | (Masked | |
| Patient# | Side) | Side) | Side) | Side) | Average |
| 1 | 10 | 10 | 10 | 10 | 10 |
| 2 | 7 | 7 | 7 | 7 | 7 |
| 3 | 9 | 9 | 9 | 9 | 9 |
| 4 | 7 | 7 | 7 | 7 | 7 |
| 5 | 10 | 10 | 10 | 10 | 10 |
| 6 | 8 | 8 | 8 | 8 | 8 |
| 7 | 8 | 8 | 8 | 8 | 8 |

| | Numbness- | Numbness | Numbness- | Numbness- | |
|---|---|---|---|---|---|
| | Mouth | Cheeks- | Forehead | Chin | |
| | (Unmasked | (Unmasked | (Unmasked | (Unmasked | Aver- |
| Patient# | Side) | Side) | Side) | Side) | age |
| 1 | 8 | 8 | 9 | 9 | 8.5 |
| 2 | 6 | 6 | 6 | 6 | 6 |
| 3 | 6 | 6 | 6 | 6 | 6 |
| 4 | 6 | 6 | 6 | 6 | 6 |
| 5 | 8 | 8 | 8 | 8 | 8 |
| 6 | 6 | 6 | 6 | 6 | 6 |
| 7 | 8 | 8 | 8 | 8 | 8 |

| | Numbness | Numbness |
|---|---|---|
| | Average | Average |
| | Masked | Unmasked |
| Patient# | Side | Side |
| 1 | 10 | 8.5 |
| 2 | 7 | 6 |
| 3 | 9 | 6 |
| 4 | 7 | 6 |
| 5 | 10 | 8 |
| 6 | 8 | 6 |
| 7 | 8 | 8 |

Table XII shows results of the pain level of felt by patients using the mask of the invention with the mask showing lower average pain on 0-10 scale.

TABLE XII

| | | Pain After | Pain After | Pain After | Average |
|---|---|---|---|---|---|
| | Pain After | proc. Cheeks | proc. Forehead | proc. Chin | Pain |
| | proc. Mouth | (Masked | (Masked | (Masked | (Masked |
| Patient# | (Masked Side) | Side) | Side) | Side) | Side) |
| 1 | 6 | 6 | 6 | 6 | 6 |
| 2 | 5 | 5 | 5 | 5 | 5 |
| 3 | 6 | 6 | 6 | 6 | 6 |
| 4 | 1 | 0 | 1 | 1 | 0.8 |
| 5 | 3 | 3 | 3 | 3 | 3 |
| 6 | 5 | 5 | 5 | 5 | 5 |
| 7 | 5 | 5 | 5 | 5 | 5 |

| | | Pain After | Pain After | Pain After | Average |
|---|---|---|---|---|---|
| | Pain After | proc. Cheeks | proc. Forehead | proc. Chin | Pain |
| | proc. Mouth | (Unmasked | (Unmasked | (Unmasked | (Unmasked |
| Patient# | (Unmasked Side) | Side) | Side) | Side) | Side) |
| 1 | 8 | 8 | 8 | 8 | 8 |
| 2 | 8 | 8 | 8 | 8 | 8 |
| 3 | 9 | 9 | 9 | 9 | 9 |
| 4 | 2 | 2 | 2 | 2 | 2 |
| 5 | 5 | 5 | 5 | 5 | 5 |
| 6 | 8 | 8 | 8 | 8 | 8 |
| 7 | 6 | 6 | 7 | 6 | 6.3 |

| | Average | Average |
|---|---|---|
| | Pain | Pain |
| | Masked | Unmasked |
| Patient# | Side | Side |
| 1 | 6 | 8 |
| 2 | 5 | 8 |
| 3 | 6 | 9 |
| 4 | 1 | 2 |
| 5 | 3 | 5 |
| 6 | 5 | 8 |
| 7 | 5 | 6.3 |

The above described embodiments are exemplary and the claims appended hereto are not intended to be limited to the disclosure. Rather, the claims should be entitled to a breadth of scope of protection.

What is claimed is:

1. A therapeutic mask, including:
a therapeutic composition including honey and a penetrating substance;
a dressing having said therapeutic composition combined therewith, wherein said dressing and said therapeutic composition are freeze dried to provide a therapeutic mask.

2. The therapeutic mask of claim 1, wherein said therapeutic composition includes one or more of a moisture retaining substance, an emulsifier, a surfactant, a cellular repairing substance, an antioxidant, a keratin poietic, a humectant and a revitalize.

3. A method of manufacturing a therapeutic mask, the method including the steps of:
a) preparing a therapeutic composition including honey and a penetrating substance;
b) combining said therapeutic composition with a dressing material; and
c) freeze drying said combined dressing material and therapeutic composition to provide a therapeutic mask.

4. The method of claim 3, wherein said composition includes one or more of a moisture retaining substance, an emulsifier, a surfactant, a cellular repairing substance, an antioxidant, a keratin poietic, a humectant and a revitalize.

5. A method of treating skin, which includes the steps of:
(a) providing a therapeutic composition including honey and a penetrating substance, a dressing material having the therapeutic composition associated therewith, and wherein the dressing material and therapeutic composition are further characterized to be freeze dried to form the therapeutic mask;
(b) rehyrdating the mask prior to use; and
(c) placing the therapeutic mask on mammalian skin for a period of time sufficient to permit the therapeutic composition to penetrate the skin.

6. The method of claim 5, which includes the step of applying a dermal treatment prior to applying the therapeutic mask.

7. The method of claim 6, wherein the dermal treatment includes use of one of a chemical composition, CO₂ or laser.

8. The method of claim 5, which further includes the steps of removing the mask and then applying a dermal treatment.

9. The method of claim 8, wherein the dermal treatment can include at least one of an anesthetic, dermaceutical, CO₂ or laser.

10. The method of claim 8, which includes a step of reapplying a therapeutic mask as described in claim 5 subsequent to applying the dermal treatment composition.

11. The therapeutic mask of claim 1, wherein said mask is freeze dried to a moisture content of less than about 15%.

12. The therapeutic mask of claim 1, wherein said therapeutic composition includes includes honey, a penetrating substance and at least one of a moisture retaining substance, an emulsifier, a surfactant, a cellular repairing substance, an antioxidant, a keratin poietic, a humectant and a revitalizer.

13. The therapeutic mask of claim 1, wherein said therapeutic composition includes honey, a moisture retaining substance, a penetrating substance and at least one of an emulsifier, a surfactant, a cellular repairing substance, an antioxidant, a keratin poietic, a humectant and a revitalizer.

14. The therapeutic mask of claim 1, wherein said therapeutic composition includes includes honey, a penetrating substance, a cellular repairing substance and at least one of a moisture retaining substance, an emulsifier, a surfactant, an antioxidant, a keratin poietic, a humectant and a revitalizer.

15. The therapeutic mask of claim 1, wherein said therapeutic composition includes includes honey, a penetrating substance, an antioxidant and at least one of a moisture retaining substance, an emulsifier, a surfactant, a cellular repairing substance, a keratin poietic, a humectant and a revitalize.

16. The therapeutic mask of claim 1, wherein said therapeutic composition includes includes honey, a penetrating substance, a keratin poietic and at least one of a moisture retaining substance, an emulsifier, a surfactant, a cellular repairing substance, an antioxidant, a humectant and a revitalizer.

17. The therapeutic mask of claim 1, wherein said therapeutic composition includes includes honey, a penetrating substance, a humectant and at least one of a moisture retaining substance, an emulsifier, a surfactant, a cellular repairing substance, an antioxidant, a keratin poietic and a revitalizer.

18. The therapeutic mask of claim 1, wherein said therapeutic composition includes honey, a penetrating substance, a revitalizer and at least one of a moisture retaining substance, an emulsifier, a surfactant, a cellular repairing substance, an antioxidant, a keratin poietic, and a humectant.

19. The method of claim 3, wherein said mask is freeze dried to a moisture content of less than about 15%.
